(19) United States
(12) Patent Application Publication
(10) Pub. No.: US 2026/0288802 A1
(43) Pub. Date: Sep. 24, 2026

(54) SYSTEM

(71) Applicant: SoftBank Group Corp., Tokyo (JP)

(72) Inventor: Daisuke YAMAGATA, Tokyo (JP)

(73) Assignee: SoftBank Group Corp., Tokyo (JP)

(21) Appl. No.: 19/564,640

(22) Filed: Mar. 12, 2026

(30) Foreign Application Priority Data
Mar. 19, 2025 (JP) 2025-044961

(51) Int. Cl. G06F 16/3329; G06F 18/2433; G06N 3/0499; G16H 10/60

(52) U.S. Cl. CPC G06F 16/33295; G06F 18/2433; G06N 3/0499; G16H 10/60

(57) ABSTRACT

A system includes a processor that is configured to: convert biometric information into digital data by using a sensor device for inputting the biometric information, transmit the digital data to a server via the Internet, cause the server to store the digital data in a database, analyze the digital data by using a statistical method, and generate a prompt for instructing a generative AI model to generate feedback based on an analysis result of the digital data, and cause the generative AI model to generate the feedback and cause the feedback to be displayed on a terminal of a user.

### CROSS-REFERENCE TO RELATED APPLICATION

This application is based on and claims priority under 35 USC 119 from Japanese Patent Application No. 2025-044961 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional health management systems that handle biometric information mainly perform simple monitoring or threshold-based alerting and provide static guidance to a user. In such systems, biometric information collected by a sensor device is often stored and displayed without being subjected to advanced analysis, and even when some statistical processing is performed, feedback to the user is typically limited to generic messages that are not sufficiently personalized. As a result, it is difficult for the user to obtain concrete and individualized action guidelines that are directly linked to improvement of a mental and physical health condition. Furthermore, existing systems generally do not utilize generative AI models in a systematic manner to transform analyzed biometric data into rich, context-aware feedback, and do not effectively combine statistical analysis with natural language processing to generate feedback that is easily understandable by the user. Accordingly, there is a need for a system capable of converting biometric information into digital data, analyzing the digital data by using statistical methods, and then, by utilizing a generative AI model, generating and presenting to the user personalized feedback including specific action guidelines for improving the mental and physical health condition.

### SUMMARY

In order to solve the above-described problem, according to one aspect of the present invention, there is provided a system comprising a processor, wherein the processor is configured to convert biometric information into digital data by using a sensor device for inputting the biometric information, transmit the digital data to a server via the Internet, and cause the server to store the digital data in a database. The processor is further configured to cause the server to analyze the digital data by using a statistical method and generate a prompt for instructing a generative AI model to generate feedback based on an analysis result of the digital data, and to cause the generative AI model to generate the feedback and cause the feedback to be displayed on a terminal of a user. The processor is also configured to cause the generative AI model to analyze the prompt by using a natural language processing technique and generate the feedback based on information related to a mental and physical health condition. In addition, the processor is configured to cause the feedback to include a concrete action guideline for improvement of the mental and physical health condition and to be customized in consideration of a past data history of the user. By this configuration, the system can automatically transform raw biometric information into statistically analyzed results and, through the generative AI model, provide the user with individualized and actionable feedback that supports improvement and management of the mental and physical health condition.

The term “system” refers to an arrangement including at least one processor and one or more associated devices, servers, terminals, databases, and software components that cooperatively perform the functions described in the claims.

The term “processor” refers to any hardware or combination of hardware and software capable of executing instructions, such as a CPU, microcontroller, FPGA, GPU, or system-on-chip, that performs the processing operations described in the claims.

The term “biometric information” refers to physiological or biological data of a user, including, but not limited to, heart rate, blood pressure, body temperature, respiration rate, sleep data, activity level, or other measurable indicators of a mental or physical health condition.

The term “sensor device” refers to any device configured to measure biometric information of a user and convert the measured biometric information into an electrical signal, including, but not limited to, wearable sensors, medical monitoring devices, smartphones with integrated sensors, or external measurement instruments.

The term “digital data” refers to data representing biometric information in a machine-readable digital format suitable for storage, transmission, and processing by the system.

The term “server” refers to one or more computing devices, which may be local or remote and may be implemented as physical machines, virtual machines, or cloud-based instances, that store, analyze, and process digital data and interact with the generative AI model.

The term “Internet” refers to a global network of interconnected communication systems enabling data exchange between the processor, the server, and other components by means of standard communication protocols.

The term “database” refers to any structured or semi-structured data storage system, including relational databases, NoSQL databases, or other data repositories, that stores digital data associated with biometric information and analysis results.

The term “statistical method” refers to any algorithm, technique, or procedure that applies statistical analysis to digital data, including, but not limited to, calculation of averages, variances, trends, correlations, classifications, clustering, or time-series analysis.

The term “analysis result” refers to information derived from digital data by applying a statistical method, including aggregate values, patterns, trends, anomalies, or indicators of a mental or physical health condition.

The term “generative AI model” refers to an artificial intelligence model capable of generating new content, such as text or other media, based on an input prompt and trained on data sets, including but not limited to large language models and other generative models.

The term “prompt” refers to a data structure, typically expressed in natural language or a structured format, that encodes instructions, constraints, or context for the generative AI model to generate feedback based on the analysis result.

The term “feedback” refers to an output generated by the generative AI model, including explanations, suggestions, or recommendations, that is intended to inform the user about a mental or physical health condition and provide guidance for improvement or management.

The term “terminal” refers to any user-operated device capable of communicating with the server and displaying feedback, including, but not limited to, smartphones, tablet computers, personal computers, wearable devices, or dedicated health monitoring terminals.

The term “natural language processing technique” refers to any algorithm, model, or method that enables the generative AI model to interpret, understand, and generate human language, including parsing, semantic analysis, context handling, and language generation.

The term “mental and physical health condition” refers to a state of health of the user that includes both psychological aspects, such as stress, mood, or fatigue, and physical aspects, such as cardiovascular status, sleep quality, or activity level.

The term “concrete action guideline” refers to specific, actionable instructions included in the feedback, such as recommended behaviors, lifestyle changes, exercises, or medical consultations, that the user can practically perform to improve the mental and physical health condition.

The term “past data history” refers to previously collected digital data and associated analysis results for a particular user, stored in the database and used to personalize or customize subsequent feedback generated by the system.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

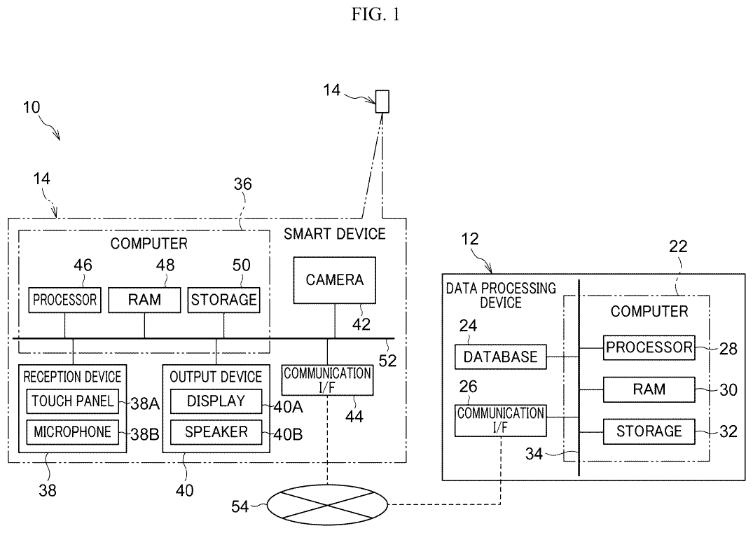

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

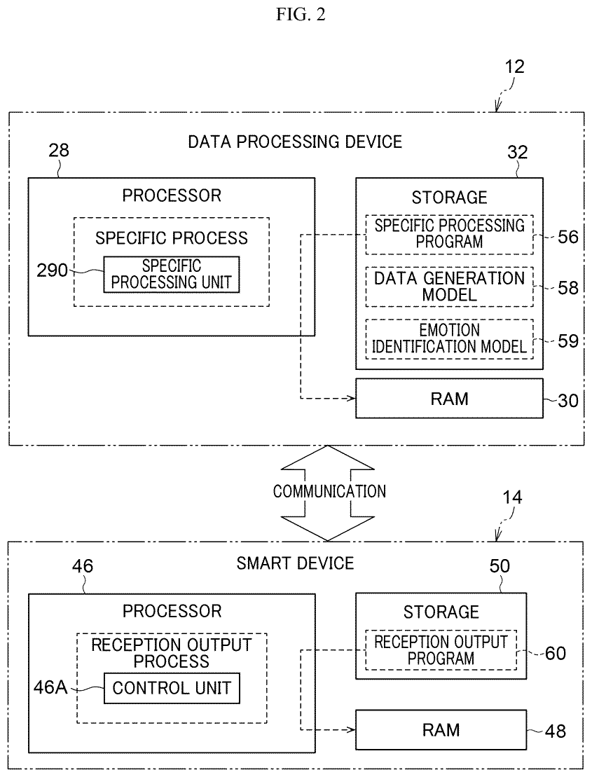

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented health monitoring systems typically transmit raw biological measurements from a sensor device to a server, apply simple rule-based or threshold-based checks, and then present generic recommendations to the user. In such systems, the server often processes each data point in isolation, without efficiently exploiting longer-term historical data, statistical trend analysis, or structured health state evaluation. As a result, the generated feedback is coarse-grained, not well personalized, and frequently redundant. From a computer-technology standpoint, these approaches underutilize available processing resources and data structures, because they neither organize heterogeneous biological information and historical records into a unified evaluation representation, nor leverage such representation to control an advanced language generation component in a structured and auditable way.

Furthermore, in many existing architectures, integration between a statistical analysis engine and a generative AI model is loosely defined. Prompt sentences for the generative AI model are often constructed in an ad hoc manner at the application layer, without a standardized internal format that encodes computed statistics, anomaly detection results, and temporal trends. This leads to unstable system behavior, because small changes in prompt wording may cause large variations in output, and the system cannot reliably reproduce or audit prior outputs. The absence of explicit linkage between stored biological data, derived evaluation data, and generated feedback also hampers traceability, quality control, and systematic improvement of the model interaction.

In addition, prior systems commonly treat the generative AI model as a black-box text generator operating on raw user descriptions, rather than as a component tightly orchestrated by the server's data-processing pipeline. Without a structured, machine-generated prompt that embeds quantitative evaluation, user attributes, and trend indices, the generative AI model cannot effectively exploit the full richness of the available data. This results in inefficient utilization of computational resources on both the server and the model side, and prevents the system from generating consistently customized feedback that is sensitive to temporal changes in a user's health-related patterns.

Accordingly, there is a need for an improved computer-implemented system and processing method in which a processor (i) converts biological information into structured digital data, (ii) executes statistical and time-series analysis over historical records to generate a machine-interpretable health state evaluation, (iii) constructs a standardized prompt sentence embedding this evaluation, user attributes, and trend indicators, and (iv) uses this prompt sentence to control a generative AI model. Such a system should further record explicit correspondences among the underlying data, the constructed prompt, and the generated feedback, thereby improving traceability, reproducibility, and overall reliability of the computer-implemented health feedback generation process.

The specific processing by the specific processing unit 290 of the data processing device 12 in Example 1 is realized by the following means.

The present invention provides a server comprising a processor configured to acquire biological information from a detection apparatus, convert the biological information into digital data, and store the digital data as structured records in a storage device; to retrieve the stored digital data together with historical records associated with a user, and execute statistical analysis processing including calculation of statistical values, time-series analysis, and anomaly detection to generate health state evaluation data for the user; to construct a prompt sentence as structured input information including the health state evaluation data, user attribute information, and trend indicators derived from the historical records, the prompt sentence being configured to instruct a generative AI model to generate natural-language feedback; to transmit the prompt sentence to the generative AI model and receive natural-language feedback information generated by the generative AI model; and to transmit output data based on the feedback information to a user terminal while storing, in the storage device, a correspondence among the feedback information, the prompt sentence, and the health state evaluation data. This enables a computer-implemented health monitoring system in which server-side data structures, statistical processing, and generative AI control are technically integrated so that the generative AI model operates on a machine-generated, standardized prompt embedding quantitative evaluation and temporal trends, thereby improving the efficiency, reproducibility, personalization, and traceability of feedback generation as a whole computer technology.

The term “biological information” refers to data indicative of a physical or mental condition of a living subject, including but not limited to heart rate, blood pressure, sleep duration, physical activity, dietary information, and stress-related indicators. The term “detection apparatus” refers to any hardware configured to sense, measure, or otherwise obtain biological information from a subject and output the information as electrical or optical signals, such as a sensor device, wearable device, or measuring instrument.

The term “digital data” refers to biological information represented in a discrete, machine-readable format suitable for processing, storage, and transmission by an electronic computing system.

The term “communication network” refers to a wired or wireless data-communication infrastructure over which digital data can be transmitted between devices, including local networks and wide-area networks such as the Internet.

The term “information processing apparatus” refers to an electronic computing system, such as a server device or cloud-based processing node, configured to receive, store, and process digital data.

The term “storage device” refers to any physical or virtual data-storage component of an information processing apparatus, including volatile and non-volatile memory, configured to retain structured data, historical records, and generated feedback.

The term “structured data” refers to digital data organized according to a predefined logical format, such as a record, table, or schema, enabling indexing, querying, and statistical processing by an information processing apparatus.

The term “historical data” refers to previously stored digital data and related records associated with a particular user or subject over a past time period, including past biological information and derived evaluation results.

The term “statistical analysis processing” refers to computational operations that apply mathematical or statistical techniques to digital data, including calculation of statistical values, time-series analysis, and anomaly detection.

The term “statistical values” refers to numerical indicators derived from digital data, such as averages, medians, variances, standard deviations, and other statistical measures.

The term “time-series analysis” refers to processing that evaluates changes or patterns in digital data over time, including trend extraction, comparison between time intervals, and detection of temporal correlations.

The term “anomaly detection” refers to processing that identifies data points or patterns that deviate from expected or normal behavior based on statistical criteria or learned baselines.

The term “health state evaluation data” refers to digital data representing a computed assessment of a user's physical or mental condition, derived from biological information and historical data using statistical analysis processing.

The term “user attribute information” refers to data that characterizes a user independently of current measurements, including demographic information and other profile information relevant to health evaluation.

The term “trend indicators” refers to quantitative or qualitative values derived from time-series analysis that indicate changes or tendencies in biological information or health-related metrics over a predetermined period.

The term “prompt sentence” refers to structured input information, represented in natural language or a combination of natural language and machine-readable format, that encodes health state evaluation data, user attribute information, and trend indicators, and is supplied to a generative AI model to instruct generation of feedback.

The term “generative AI model” refers to a machine-implemented model configured to generate output data, including natural-language text, in response to input such as a prompt sentence, and implemented using a statistical or neural-network-based architecture.

The term “natural-language feedback information” refers to machine-generated text, expressed in a human language, that provides evaluation, explanation, or recommendations regarding a user's health-related state in response to a prompt sentence.

The term “output data” refers to digital data derived from natural-language feedback information and formatted for presentation on a user terminal, including text, metadata, and layout information.

The term “user terminal” refers to an end-user electronic device configured to communicate with the information processing apparatus and present output data to a user, such as a mobile device, computing device, or display apparatus.

The term “display unit” refers to a component of a user terminal that visually presents information to a user, including screens, panels, or other image-display devices.

The term “correspondence” refers to an explicit association recorded in the storage device that links feedback information with the prompt sentence and the health state evaluation data from which the prompt sentence was constructed.

In one embodiment, a server, a terminal, and a user cooperate to implement the present invention. The server is realized as a computing device including at least one central processing unit (CPU), a main memory, a non-volatile storage device, and a network interface connected to a communication network such as the Internet. The server executes an operating system and application software including a web-application framework, a data-analysis module, a model-interface module for a generative AI model, and a database management system. The terminal is realized as a mobile computing device, such as a smartphone, including a processor, a memory, a display unit, an input unit (touchscreen), and communication interfaces including wireless communication modules. The user operates the terminal and optionally wears a sensor apparatus, such as a wearable device having one or more biological sensors.

The terminal uses sensor interfaces to cooperate with a detection apparatus that measures biological information. The terminal utilizes a wireless communication module, such as a short-range radio communication interface, to connect to the detection apparatus. The terminal thereby obtains biological information represented as measurement values, such as heart-rate samples, step counts, and sleep intervals. The terminal also provides graphical user interface screens implemented with a user interface framework, enabling the user to manually input biological information including blood pressure values, daily exercise time, meal summaries, and self-reported stress levels. The terminal converts both automatically obtained measurements and manually entered values into digital data in a standardized internal format, for example records having fields for user identifier, measurement type, numerical value, unit, and timestamp.

The terminal stores the digital data temporarily in a local data store, such as a lightweight relational database managed by a mobile persistence framework. The terminal marks each locally stored record with a synchronization status flag indicating whether the record has been transmitted to the server. The terminal compresses multiple unsent records into a batched payload and encodes the payload into a structured textual representation, such as a delimited format, and transmits the payload to the server via a secure communication protocol using a network stack of the operating system. By batching records and reusing a single connection for multiple measurements, the terminal reduces communication overhead and improves transmission efficiency.

The server receives the transmitted digital data via the network interface and a web-application framework. The server authenticates the terminal using a token contained in a header field and validates the payload according to a schema that defines permitted measurement types, numerical ranges, and required fields. The server stores the validated digital data as structured data in a database system, such as a relational database, by mapping each measurement to a row in a table having columns for user identifier, measurement type, measurement value, measurement unit, and measurement time. The server also stores index structures on user identifier and time to accelerate later queries over historical data. The server maintains a separate table for derived evaluation data and for generated feedback text, and records explicit foreign-key relations among measurements, evaluations, and feedback entries.

The server uses a data-analysis module implemented in a programming language, together with a numerical library such as an array-processing library and a tabular-data library, to process the structured digital data. The server retrieves, for a given user, a time window of historical measurements for each measurement type, such as the latest 30 days of sleep duration records or heart-rate measurements. The server loads these records into in-memory data structures, such as columnar arrays, and computes statistical values including arithmetic mean, minimum, maximum, variance, and standard deviation for each measurement type. The server further performs time-series analysis by computing moving averages over predefined windows, difference series, and trend indicators, such as linear regression slopes of average values over time. The server executes anomaly detection by computing normalized deviation values, for example a z-score for the latest measurement relative to the historical distribution, and by comparing the normalized deviation to a threshold value stored as a configuration parameter.

The server aggregates the computed statistics, anomaly flags, and trend indicators into health state evaluation data. The server encodes this evaluation data as structured records that link, for each metric, a numerical summary (e.g., mean sleep hours, change percentage week-over-week), a categorical label (e.g., “within expected range,” “slightly high,” “significantly low”), and a trend classification (e.g., “increasing,” “decreasing,” “stable”). The server stores these evaluation records in the database, referencing both the user and the underlying measurements. By doing so, the server creates a reusable, machine-interpretable representation of the user's health state that can be efficiently queried and compared over time.

The server constructs a prompt sentence for a generative AI model by programmatically combining the health state evaluation data with user attribute information and trend indicators. The server uses a string-composition module to generate a textual prompt sentence in natural language that follows a predetermined pattern. For example, the server may generate a prompt sentence such as:
“User: male in his 30s. Recent biometric data: resting heart rate 72 bpm, blood pressure 125/82, sleep duration 6.5 hours, daily exercise 40 minutes (mostly moderate-intensity walking), meals often late at night and moderately high in salt, self-reported stress level 8/10. Past 30-day trend: sleep duration has decreased by about 1 hour, resting heart rate has increased by about 4 bpm, and average systolic blood pressure is slightly higher than the previous month but still within a normal range. Task: As a health coach, evaluate the user's current physical and mental health status and provide specific, practical advice for improving sleep quality, managing stress, and maintaining cardiovascular health. Avoid making medical diagnoses. Provide the output in Japanese and keep it within approximately 300 words.”

In another example, the server may generate a prompt sentence such as:
“A 45-year-old female user has the following daily health data: average heart rate 68 bpm, blood pressure 118/76, sleep time 7.5 hours, daily walking 9,000 steps, stress level reported as 3/10, and meals described as balanced with vegetables at every meal. Over the past 60 days, there are no significant anomalies or negative trends. Please generate a friendly summary of her health status and three concrete suggestions to maintain or slightly improve her current lifestyle. Write the answer in English at a middle-school reading level, and keep it under 200 words.”

The server thereby ensures that the prompt sentence embeds structured, quantitative evaluation and trend information in a standardized textual form. The server uses a model-interface module to send the prompt sentence to a generative AI model. The generative AI model is realized as a trained neural-network model, such as a transformer-based sequence model including multiple layers of self-attention and feed-forward sublayers. The server supplies the prompt sentence to the generative AI model via an application programming interface, together with configuration parameters such as a temperature parameter controlling output randomness and a maximum token count limiting response length.

The server causes the generative AI model to perform internal computations on the prompt sentence. The generative AI model tokenizes the prompt sentence into discrete tokens according to a tokenizer trained together with the model. The generative AI model associates each token with an embedding vector and processes sequences of embedding vectors through a plurality of transformer layers. Each transformer layer computes attention scores over the sequence of tokens using learned weight matrices and applies nonlinear transformation functions to compute updated hidden representations. The generative AI model generates, token by token, a sequence of output tokens representing natural-language feedback. The model has been pre-trained on large-scale textual data and fine-tuned on instruction-following tasks, using an optimization method such as stochastic gradient descent with an adaptive optimizer, and a loss function such as cross-entropy between predicted token distributions and target sequences.

The server receives the generated output tokens from the generative AI model and decodes them into a feedback text. The server optionally performs post-processing, such as truncating excessive length, removing disallowed phrases according to a pattern-matching module, and enforcing inclusion of certain structural elements such as a short summary followed by bullet-like recommendations. The server then stores the feedback text, together with the corresponding prompt sentence and health state evaluation data identifier, in the database. By recording this explicit correspondence, the server enables later auditing of which input data and which evaluation results led to which feedback.

The server transmits the processed feedback text to the terminal. The server encodes the feedback text, along with metadata including a timestamp and an overall risk level indicator, into a response message and sends it to the terminal via the communication network. The server may additionally send a notification request to a push-notification service, causing the terminal to present a notification if the application is not currently in the foreground. The terminal receives the feedback text and uses a rendering module to update the display unit. The terminal renders the feedback text as human-readable content with sections such as “Overall Status,” “Sleep,” “Exercise,” “Diet,” and “Stress,” highlighting metrics classified as abnormal in a visually distinct manner.

The user views the feedback on the terminal and may adjust behavior according to the recommendations. The user can scroll through previous feedback entries, which the terminal obtains from the server or from a locally cached copy. The user thereby perceives longitudinal changes in evaluation and receives explanations that explicitly reference trend information, such as “Your average sleep time has decreased for three consecutive weeks.”

The server thus improves computer-technology aspects of health feedback generation in several concrete ways. The server reduces communication overhead and storage redundancy by storing raw measurements once and computing separate, compact health state evaluation data for prompt construction, rather than repeatedly sending large unstructured descriptions to the generative AI model. The server improves processing efficiency by using specialized numerical libraries and pre-computed indices to perform statistical and time-series analysis on structured data, reducing query latency for each evaluation. The server improves output accuracy and stability by embedding standardized trend indicators and quantitative summaries into the prompt sentence according to a fixed template, which reduces variance in model outputs caused by inconsistent free-form inputs. The server enhances traceability and debuggability by storing correspondences among measurements, evaluation data, prompt sentences, and feedback texts, enabling systematic analysis of model behavior and iterative refinement of the prompt design.

The server further separates responsibilities between deterministic numerical computation and probabilistic language generation. Statistical analysis and anomaly detection are executed by deterministic algorithms running on the server's CPU, using explicit thresholds, moving-window computations, and regression-based trend estimation. The generative AI model receives as input not raw sensor data but the already processed health state evaluation data, which concentrates information into higher-level features. This division allows the system to use the generative AI model's computational capacity more efficiently for natural-language expression and explanation, rather than for low-level pattern extraction that is already handled by the server's analytical pipeline.

In additional embodiments, the server may implement different statistical or machine-learning algorithms for anomaly detection, such as clustering-based outlier identification or state-space modeling for time-series trends. The server may select among multiple generative AI models with different sizes or latency characteristics depending on resource conditions or the required detail level of the feedback. The terminal may be realized not only as a mobile device but also as a stationary computing device or a specialized health-monitoring display terminal. Likewise, the detection apparatus may include various sensor types, such as optical sensors, mechanical sensors, or electrodes, provided that the apparatus outputs biological measurements in a format that can be converted into digital data by the terminal or the server.

Through these structural elements and processing operations, the server, the terminal, and the user cooperate to implement a system that does more than automate a human counselor's activity. The system exploits structured digital data, deterministic numerical analysis, and a transformer-based generative AI model guided by carefully constructed prompt sentences, thereby improving computation efficiency, communication efficiency, and the technical quality of generated feedback within a computer-implemented health monitoring environment.

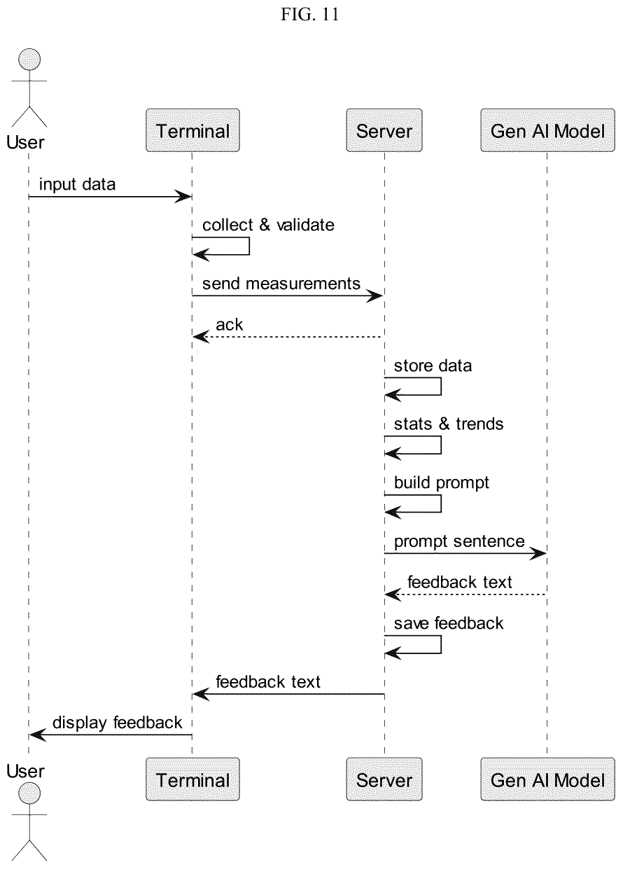

The following describes the processing flow using FIG. 11.

### Step 1:

The user operates the terminal to provide biological information. The user manually inputs values such as heart rate, blood pressure, sleep duration, exercise time, meal description, and stress level into input fields on the terminal display, or confirms automatically acquired values from a detection apparatus. The input of this step is raw user readings and confirmations, and the output is a set of typed values and selections held in the terminal's working memory in a temporary data structure.

### Step 2:

The terminal acquires additional biological information from a detection apparatus. The terminal uses a wireless interface to connect to the detection apparatus and reads sensor measurements such as periodic heart-rate samples, step counts, and sleep intervals. The input of this step is low-level sensor signals or device-provided measurement records, and the terminal converts them into digital data records including user identifier, measurement type, value, unit, and timestamp. The output is a collection of normalized measurement records ready for local storage.

### Step 3:

The terminal aggregates and validates the biological information. The terminal merges the manually entered values and the automatically obtained measurements into a unified in-memory list or array, then checks each record for valid ranges, correct types, and missing fields. The input of this step is the separate sets of raw user inputs and sensor measurements, and the terminal runs data-validation logic and simple numerical checks (for example, heart rate>0 and within a plausible upper bound) to produce, as output, a cleaned and consolidated set of measurement records.

### Step 4:

The terminal stores the biological information in a local data store. The terminal writes each validated record as a row in a local table managed by a mobile database engine, and sets a synchronization status field to indicate that the record has not yet been sent to the server. The input of this step is the cleaned list of measurement records from the previous step, and the output is a persistent local dataset with each record tagged by synchronization status and indexed by timestamp and measurement type.

### Step 5:

The terminal prepares a transmission batch for the server. The terminal queries the local data store for all records whose synchronization status indicates “unsent,” packages these records into a batched payload, and encodes them in a structured textual format. The input of this step is unsent local records, and the terminal processes them by grouping, serializing, and compressing where applicable so that the output is a compact batch payload suitable for network transmission.

### Step 6:

The terminal transmits the batched digital data to the server. The terminal opens a secure network connection using a transport protocol with encryption, attaches an authentication token in the header, and sends the batch payload as the body of a request. The input of this step is the batched payload and authentication information, and the terminal uses the operating system's networking stack to encapsulate, encrypt, and transmit the data, producing as output a network message delivered to the server.

### Step 7:

The server receives and authenticates the digital data. The server accepts the network message through a network interface and a web-application framework, verifies the authentication token, and checks that the payload conforms to a predefined schema. The input of this step is the encrypted network message, which the server decrypts and parses, and the output is a validated in-memory representation of the measurement batch or an error response if validation fails.

### Step 8:

The server stores the measurement records as structured data. The server maps each measurement in the validated batch to a row in one or more database tables, filling columns such as user identifier, measurement type, measurement value, unit, and measurement time. The input of this step is the set of validated measurement records in memory, and the server executes database insert operations and index updates, producing as output a persistent, queryable measurement dataset in the storage device.

### Step 9:

The server retrieves historical data for analysis. The server issues database queries filtered by user identifier and by a predefined time window (for example, the most recent 30 or 60 days) and obtains, for each measurement type, a chronological series of values. The input of this step is the user identifier and time-window parameters, and the server performs indexed lookups and sorts to output ordered sequences of measurements grouped by type, ready for numerical computation.

### Step 10:

The server performs statistical computations on the historical and current data. The server loads the retrieved sequences into in-memory numerical arrays and computes, for each measurement type, statistics such as mean, minimum, maximum, variance, and standard deviation, and also calculates moving averages and differences between consecutive periods. The input of this step is the grouped time-series data from the database, and the server applies numerical operations and algorithms to produce, as output, a table of statistical values and intermediate time-series results.

### Step 11:

The server executes time-series trend analysis and anomaly detection. The server uses the statistical values and time-series data to compute trend indicators, such as regression slopes or percentage changes over defined intervals, and calculates normalized deviation values (for example, z-scores) for the latest measurements. The input of this step is the computed statistics and ordered sequences of values, and the server compares the normalized deviations against configured thresholds and classifies each metric as “within expected range,” “slightly abnormal,” or “significantly abnormal.” The output is a set of trend indicators and anomaly flags for each measurement type.

### Step 12:

The server generates health state evaluation data. The server combines the statistics, trend indicators, and anomaly flags into evaluation records that summarize the user's condition per metric and overall. The input of this step is the numerical and categorical results from trend and anomaly analysis, and the server constructs structured evaluation entries with fields such as summary value, categorical label, and trend description. The output is health state evaluation data stored in memory and optionally written to a dedicated evaluation table in the database.

### Step 13:

The server composes a prompt sentence for a generative AI model. The server retrieves user attribute information and selects relevant parts of the health state evaluation data and trend indicators, then inserts these values into a predefined natural-language template. The input of this step is the evaluation data and user attributes, and the server performs string formatting and concatenation operations to produce, as output, a textual prompt sentence that encodes the user profile, current measurements, historical trends, and requested response style.

### Step 14:

The server transmits the prompt sentence to the generative AI model. The server calls a model interface, supplying the prompt sentence and configuration parameters such as maximum output length and temperature, via a network or local process boundary. The input of this step is the composed prompt sentence and model parameters, and the server packages them into a request message that is sent to the generative AI model, producing as output a pending inference request.

### Step 15:

The server receives natural-language feedback from the generative AI model. The generative AI model processes the prompt sentence internally and returns a sequence of output tokens forming feedback text. The input of this step, from the server's perspective, is the model's response message containing the token sequence, and the server decodes the tokens into a human-readable text string. The output is a feedback text in natural language that reflects the prompt sentence's embedded evaluation information.

### Step 16:

The server post-processes and stores the feedback text. The server analyzes the generated feedback text for length, prohibited expressions, and required structural elements, optionally removes or replaces parts according to rule-based filters, and then associates the cleaned feedback with the corresponding health state evaluation data and prompt sentence. The input of this step is the raw feedback text and references to the underlying evaluation records, and the server writes a feedback record and linkage information into the database, producing as output a stored, auditable feedback entry.

### Step 17:

The server delivers the feedback to the terminal. The server prepares a response message containing the feedback text and additional metadata such as timestamp and overall risk level and sends this message over the communication network to the terminal. The input of this step is the stored feedback text and associated metadata retrieved from memory or the database, and the output is a network response received by the terminal.

### Step 18:

The terminal presents the feedback to the user. The terminal parses the response message, extracts the feedback text and metadata, and updates user interface elements on the display unit to show the evaluation summary and detailed recommendations. The input of this step is the feedback response from the server, and the terminal performs text rendering and layout operations, producing as output a visual representation of the natural-language feedback on the screen, which the user can read and use as a basis for future health-related decisions.

Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health monitoring systems primarily focus on collecting biometric data and performing simple threshold-based evaluations on the collected data. In many implementations, a server stores time-series biometric data and applies static rule sets or basic statistical analysis to detect abnormal values. These systems suffer from several technical limitations from the perspective of computer technology.

First, conventional systems lack an integrated mechanism that combines machine learning-based anomaly detection with adaptive natural language feedback generation in a structured, machine-controlled loop. Typically, anomaly detection and feedback generation are implemented as disjoint modules or are manually operated, which results in fragmented data flows, duplicated processing, and increased latency. This separation prevents efficient utilization of computational resources and leads to inconsistent decision-making across the system.

Second, existing architectures often do not optimize the internal representation of biometric data and derived features for anomaly detection. For example, biometric data may be stored as unstructured logs or heterogeneous records, requiring repeated pre-processing and conversion whenever a model is executed. This causes redundant computation, increases memory access overhead, and degrades the responsiveness of anomaly detection processes, especially when real-time or near-real-time monitoring is required.

Third, many systems rely on fixed message templates or static content for user feedback, without constructing a context-aware prompt that exploits specific anomaly characteristics, severity levels, temporal patterns, and user attributes. As a result, even if a generative model is used, it often receives incomplete or non-optimized input, which leads to generic, non-personalized, and sometimes ambiguous feedback. From a technical standpoint, the absence of a standardized, machine-generated prompt structure limits the ability of the generative model to produce high-quality outputs and reduces the overall effectiveness of the computational pipeline.

Fourth, conventional systems do not tightly couple the anomaly detection results with the downstream communication mechanisms, such as automated notification to third-party contacts and presentation of feedback to user terminals. Often, these steps are triggered by separate applications or scripts, which require manual configuration and can lead to race conditions, inconsistent states, or delayed alerts. This fragmentation complicates system integration, increases the risk of failures, and reduces reliability of the overall computer-implemented monitoring solution.

Fifth, traditional designs do not explicitly encode, within the server logic, the generation and management of prompt sentences as first-class data objects that are dynamically constructed based on machine-learned anomaly features and historical biometric information. Without such a mechanism, it is difficult to maintain a clear, auditable mapping between numerical anomaly signals and the textual instructions given to the user, which in turn limits transparency and hinders further optimization of the computational models.

Accordingly, there is a need for a computer-implemented system and server architecture that (i) efficiently ingests biometric data into structured data representations, (ii) performs machine learning-based anomaly determination in a consistent and resource-efficient manner, (iii) dynamically generates structured prompt sentences that encode anomaly types, severities, and temporal features, and (iv) seamlessly orchestrates a generative model and communication components to produce and deliver context-aware feedback and alerts. Such a system should improve the technical performance of the health monitoring pipeline, including data processing efficiency, consistency of decisions, latency of anomaly response, and quality of automatically generated feedback, thereby providing a concrete improvement in computer technology as applied to health monitoring and alerting systems.

The specific processing by the specific processing unit 290 of the data processing device 12 in Application Example 1 is realized by the following means.

The present invention provides a server comprising a processor configured to receive biometric data converted into digital data from a detection apparatus via a communication network, store the digital data in a storage unit in a structured, table-type data representation, execute anomaly determination processing on the digital data using a machine learning processing unit to determine presence or absence of an abnormal value, automatically construct a machine-readable prompt sentence that encodes a type and severity of the abnormal value and temporal feature values derived from historical biometric information, input the prompt sentence into a generative information processing model to obtain feedback information including an action guideline corresponding to the abnormal value, and control a communication interface to (i) transmit warning information to a third-party contact when the abnormal value is detected and (ii) provide the feedback information and the warning information to a user terminal for display. This enables an integrated, computer-implemented health monitoring pipeline in which biometric data ingestion, machine learning-based anomaly detection, dynamic prompt generation, generative-model-based feedback generation, and automated notification are orchestrated within a single server-controlled architecture, thereby reducing redundant data transformations, improving processing efficiency and response latency, and enhancing the technical quality and contextual relevance of feedback delivered to user terminals and third-party contacts.

The term “biological information” refers to measurable physiological or psychological parameters of a living subject, including but not limited to heart rate, blood pressure, respiratory rate, body temperature, and other vital signs, which can be acquired by a sensor or detection apparatus and converted into digital data.

The term “detection apparatus” refers to a hardware device configured to measure biological information of a user using one or more sensors and to convert the measured information into digital data suitable for transmission to a server or other information processing apparatus.

The term “digital data” refers to information representing measured biological information in a discrete, machine-readable format, such as numerical values or encoded records, that can be processed, stored, and transmitted by electronic computing equipment.

The term “communication network” refers to a wired or wireless data transmission infrastructure, including local area networks, wide area networks, and public networks, that enables digital data to be communicated between a detection apparatus, a server, and a terminal.

The term “information processing apparatus” refers to an electronic computing environment, including at least one processor and at least one memory, configured to receive, store, and process digital data according to executable instructions.

The term “storage unit” refers to a memory device or memory subsystem, such as a semiconductor memory or magnetic storage, configured to store digital data, model parameters, and intermediate processing results in a persistent or semi-persistent manner.

The term “table-type data structure” refers to a structured arrangement of data elements organized in rows and columns, such as a data table or data frame, in which each row represents a record and each column represents a field or feature of the record, enabling efficient indexing, querying, and analysis.

The term “machine learning processing unit” refers to a functional component, implemented in hardware, software, or a combination thereof, configured to execute a machine learning algorithm or model to perform tasks such as anomaly determination, classification, regression, or clustering on input digital data.

The term “anomaly determination processing” refers to computational operations performed by the machine learning processing unit to evaluate input digital data, detect deviations from a learned normal pattern, and determine presence or absence of an abnormal value according to a model or algorithm.

The term “abnormal value” refers to a value or set of values of biological information that deviates from a normal or expected range, pattern, or distribution as determined by the anomaly determination processing, and that is associated with a potential risk or atypical condition.

The term “notification processing unit” refers to a functional component, implemented in hardware, software, or a combination thereof, configured to generate and transmit notification messages, including warning information, to external communication destinations based on detection of an abnormal value.

The term “short text message” refers to a character-based communication payload, such as a text message or similar short-form communication, that can be transmitted via a messaging service, mobile network, or data network to a recipient terminal associated with a third-party contact.

The term “third-party contact” refers to a communication destination, such as an emergency contact, caregiver, or monitoring service, registered in association with a user and configured to receive warning information when an abnormal value is detected.

The term “portable information terminal” refers to a user-operated, portable electronic device, such as a smartphone, tablet, or wearable terminal, that includes at least a processor, a communication interface, and a display device, and is capable of presenting warning information and feedback information to the user.

The term “display device” refers to a visual output component of the portable information terminal, such as a liquid crystal display, organic light-emitting diode display, or other screen, configured to present text, graphics, or user interface elements.

The term “warning information” refers to data indicating that an abnormal value has been detected, including at least a notification that a potential anomaly exists and optionally including summary information about the type or severity of the anomaly.

The term “history information of the biological information” refers to time-series or accumulated records of past biological information of a user, including measured values, timestamps, and optionally derived features, stored over a period of time for use in analysis and model input.

The term “prompt sentence” refers to a structured natural language or semi-natural language text string generated by the server, which encodes information about an abnormal value, severity, temporal features, and contextual factors, and is provided as input to a generative information processing model to elicit feedback information.

The term “generative information processing model” refers to a machine learning model, such as a generative language model, configured to receive a prompt sentence and generate text output, including feedback information, by probabilistically modeling sequences of tokens or symbols.

The term “feedback information” refers to machine-generated content produced by the generative information processing model in response to a prompt sentence, the content including at least an action guideline corresponding to an abnormal value and optionally including additional advice or explanatory information.

The term “action guideline” refers to instructions or recommendations, represented in natural language, that specify one or more steps or behaviors a user should follow in response to an abnormal value or detected condition.

The term “natural language processing technology” refers to computational techniques and algorithms that enable a system to analyze, interpret, and generate human language text, including tokenization, parsing, semantic analysis, and text generation.

The term “type of the abnormal value” refers to a classification or category that characterizes the nature of the detected anomaly, such as elevated heart rate, low heart rate, high blood pressure, or other specific anomalous condition.

The term “severity of the abnormal value” refers to a level or degree indicating how critical or urgent the abnormal value is, based on one or more criteria such as magnitude of deviation from normal, model-derived anomaly score, or duration of the abnormal state.

The term “feature values indicating temporal changes” refers to numerical or symbolic values derived from sequences of biological information over time, including but not limited to moving averages, rates of change, variability measures, and other time-series features used to characterize temporal patterns.

The term “context information” refers to additional information associated with a user or with biological information, such as user attribute information, environmental factors, or historical anomaly patterns, which may be supplied to the generative information processing model to improve relevance of feedback information.

The term “user attribute information” refers to data describing characteristics of a user, such as age, sex, known conditions, or other profile-related information, which can be utilized as part of context information for anomaly evaluation and feedback generation.

According to one embodiment, a server cooperates with a terminal and a detection apparatus to implement the claimed system. The server includes at least one processor, a main memory, a non-volatile storage device, and a network interface. The terminal includes at least one processor, a display device, a wireless communication interface, and an input device. The detection apparatus includes at least one sensor configured to measure biological information of a user, such as a heart rate sensor or a blood pressure sensor, and a wireless interface such as a short-range communication module.

The server executes an operating system and an application stack that includes a web application framework, a data processing library, a machine learning library, and a generative AI model client library. In one example, the server uses a Python runtime environment, a web framework such as a general-purpose microframework, a tabular data processing library such as a data frame library, and a machine learning library such as a general-purpose machine learning toolkit. The server stores a trained anomaly detection model and a set of parameters for a generative AI model in its non-volatile storage.

The terminal executes an application that communicates with the detection apparatus via a short-range wireless protocol such as Bluetooth Low Energy. The terminal acquires biological information, including heart rate values and blood pressure values, from the detection apparatus and converts the information into digital data structures such as key-value pairs. The terminal transmits the digital data to the server via a wide-area communication network using a secure protocol such as HTTPS. The user carries or wears the detection apparatus during daily activities so that the biological information is acquired periodically or continuously.

The server receives the digital data via the network interface and stores the data in the main memory. The server uses the data processing library to represent the digital data in a table-type data structure, for example a data frame having rows corresponding to measurement events and columns corresponding to features such as timestamp, user identifier, heart rate, systolic blood pressure, diastolic blood pressure, device identifier, and derived features. By normalizing all incoming measurement records into a consistent table-type data structure, the server reduces the need for repeated parsing and ad hoc data transformation, thereby improving data access locality and enabling efficient vectorized computation on the biological information.

The server uses the machine learning library to implement a machine learning processing unit. In one embodiment, the machine learning processing unit comprises an anomaly detection model implemented as an ensemble-based algorithm such as an isolation forest. The server trains the anomaly detection model offline using a training data set of historical biological information. During training, the server constructs feature vectors from the table-type data structure, including raw values (heart rate, systolic pressure, diastolic pressure) and temporal features such as moving averages over fixed-length windows, differences between consecutive measurements, and variability measures. The server scales the features using a normalization transform such as standardization to zero mean and unit variance.

During model training, the server defines an objective function corresponding to an anomaly detection criterion, such as minimizing a loss function that penalizes misclassification of known abnormal samples while encouraging isolation of outlying points in the feature space. The server updates internal model parameters, for example node split thresholds and feature subsets in the trees of the isolation forest, based on the training data. The training process uses multiple random subsets of features and samples to construct a plurality of decision trees, thereby producing a robust model that generalizes to unseen biological information.

In another embodiment, the machine learning processing unit uses a neural network-based anomaly detection model such as an autoencoder. The server defines an encoder-decoder architecture with multiple fully connected layers, activation functions such as rectified linear units, and a bottleneck layer representing a compressed latent space. The server trains the autoencoder on normal biological information records by minimizing a reconstruction error function such as mean squared error between the input feature vectors and the reconstructed outputs. The server updates the model parameters using an optimization algorithm such as stochastic gradient descent with adaptive learning rates. After training, the server computes an anomaly score for each new record as the reconstruction error; records with error above a threshold are treated as abnormal values. This neural network configuration enables the server to capture nonlinear correlations between different biological features, providing higher detection accuracy and lower false positives compared to simple threshold rules.

When the server receives new biological information from the terminal, the server constructs feature vectors from the table-type data structure using the same feature extraction and normalization pipeline as in training. The server passes the feature vectors to the anomaly detection model and computes anomaly scores and anomaly flags. Because the server performs these operations using vectorized operations in the data frame library and optimized routines in the machine learning library, the processing speed is higher than that of a naive implementation performing per-record ad hoc computations. This design reduces response latency, which is particularly advantageous in real-time monitoring scenarios.

When the server determines that an abnormal value is present, the server generates warning information and prepares structured information about the abnormal state. The server computes a type of the abnormal value (for example, high heart rate, low heart rate, high blood pressure) and a severity level based on the magnitude of deviation and the anomaly score. The server also computes temporal feature values indicating recent trends of the biological information, such as whether the heart rate has been increasing rapidly over the last several minutes. The server stores this information in the table-type data structure and in an alert record store. By centralizing the alert-related data in standardized structures, the server reduces overhead associated with cross-module data exchange and simplifies downstream processing.

The server then generates a prompt sentence that is to be input to a generative AI model. The server uses a prompt generation module implemented in software. This module selects fields from the table-type data structure and converts them into natural language tokens according to a defined template. For example, the server may generate a prompt sentence as follows:
“The user's heart rate has exceeded the normal range. Current heart rate: 145 beats per minute. User age: 45 years. No cardiac history is recorded in the profile. Based on recent measurements, the heart rate has been increasing over the last 10 minutes. Please provide step-by-step, easy-to-understand advice on what actions the user should take right now, and explain under what conditions the user should seek emergency medical help.”

In another example, when a high blood pressure anomaly is detected, the server may generate a prompt sentence such as:
“The user's blood pressure is above the typical healthy range. Current blood pressure: 165 over 105. The user is resting and not performing physical exercise. Previous readings in the last 30 minutes were also elevated. Give practical advice that the user can follow at home in the next 30 minutes, and indicate when the user should contact emergency medical services or a doctor.”

The prompt generation module does not simply insert values into a fixed template; instead, the module dynamically selects or omits portions of the text depending on the type and severity of the anomaly and available context, such as presence or absence of recent anomalies, user attribute information, and temporal stability of the measurements. This dynamic selection is based on conditions implemented in the server code using thresholds, rules, and dependencies derived from the anomaly determination results. By encoding structured anomaly information, severity, and temporal features into the prompt sentence, the server presents the generative AI model with a more informative and machine-optimized input, which improves the relevance and precision of the generated feedback information.

The server accesses a generative AI model provided as a neural network-based language model. In one embodiment, the generative AI model is a transformer-based architecture with multiple attention layers, trained on a large corpus of text data to predict token sequences. The server communicates with the generative AI model via an application programming interface. The server sends the prompt sentence as input tokens and specifies parameters such as maximum output length and sampling temperature. The model internally applies its neural network layers, attention mechanisms, and learned parameters to compute token probability distributions and generate a text response.

From the perspective of the server, the generative AI model operates as a deterministic software component with defined input and output formats. The server treats the model as a processing unit that transforms the prompt sentence into feedback information according to its internal parameters. Unlike a simple rule-based system or fixed template response engine, the generative AI model can synthesize nuanced instructions that depend on combinations of anomaly type, severity, and temporal patterns encoded in the prompt sentence. This enables the server to generate action guidelines that are not achievable by conventional static logic.

The server receives the feedback information generated by the generative AI model and post-processes the text. The server may, for example, split the text into bullet-like steps, remove extraneous expressions, or translate the text into a preferred language using a translation model. The server then associates the feedback information with the corresponding alert record and stores it in the storage unit.

When the server has determined that an abnormal value is present, the server also controls a notification processing unit. The notification processing unit constructs a short text message including at least minimal warning information and, optionally, a compressed summary of the anomaly. The server transmits the short text message to a third-party contact, such as a caregiver or emergency contact, using a short-message service or an equivalent communication facility. Because the server triggers third-party notification only when the anomaly determination model indicates an abnormal value and when certain severity criteria are met, the server reduces unnecessary communication traffic and avoids overloading communication networks.

The terminal periodically communicates with the server to retrieve current alert and feedback information. The server provides an application programming interface through which the terminal can request alert details associated with the user. The terminal receives structured responses and renders them on the display device. The terminal presents warning information as a message such as “Your heart rate is abnormal. A notification has been sent to your emergency contact.” The terminal also presents the generative AI model's feedback information as multi-step instructions that the user can follow. The user can acknowledge the alert or request additional details via the input device of the terminal.

The integration of the anomaly detection model, prompt generation module, generative AI model, and notification processing unit within a single server-controlled architecture yields several technical advantages. The use of the table-type data structure and vectorized feature computation reduces processing time and memory overhead, because the server performs batch operations rather than repeated scalar operations. The use of a machine learning-based anomaly detection model enables the server to detect complex patterns of abnormality in the biological information that cannot be captured by simple threshold rules, thereby reducing false alarms and missed anomalies.

The explicit encoding of anomaly type, severity, and temporal features into the prompt sentence allows the generative AI model to generate feedback information that is tightly coupled to the machine-detected anomaly. This arrangement improves the explainability and traceability of the system; the server can reconstruct, for each piece of feedback, the underlying numerical conditions that produced it. Moreover, the dynamic prompt generation logic represents a non-conventional use of generative AI models, in which the server systematically transforms structured, numeric anomaly results into natural language specifications tailored to model input requirements, thereby improving computational efficiency and content accuracy compared to naive free-form prompts.

From a computer technology perspective, the described configuration is not a mere automation of human decision making. The server uses specific data structures, algorithms, and model architectures to improve the way computers manage, analyze, and use biometric data. The server reduces redundant data parsing and transformation, optimizes anomaly detection through trained models with defined loss functions and parameter update procedures, and coordinates multiple modules in a deterministic pipeline. These design choices result in faster anomaly response times, lower processing load, better resource utilization, and higher precision of alerts and feedback.

In alternative embodiments, the server may employ different anomaly detection algorithms, such as one-class support vector machines, recurrent neural networks modeling time series, or graph-based anomaly detection. In each case, the server integrates the anomaly detection outputs into the same prompt generation and feedback delivery pipeline. The feature set may also vary; the server may include additional derived features such as circadian patterns, user activity levels obtained from motion sensors, or cross-sensor correlations. The generative AI model may be hosted on the same server or on a remote computing resource, and communication between the server and the generative AI model may occur over a dedicated secure channel.

In another variant, the terminal may perform part of the preprocessing, such as feature extraction or threshold-based pre-filtering, before sending data to the server, thereby reducing communication bandwidth. The server still centralizes anomaly detection, prompt generation, and interaction with the generative AI model. These variations remain within the scope of the claimed system, as the core technical arrangement—structured ingestion of biological information, machine learning-based anomaly determination, dynamic generation of a prompt sentence encoding anomaly features, and coordinated generation and presentation of feedback information and warning information via a generative AI model and communication interfaces—remains the same.

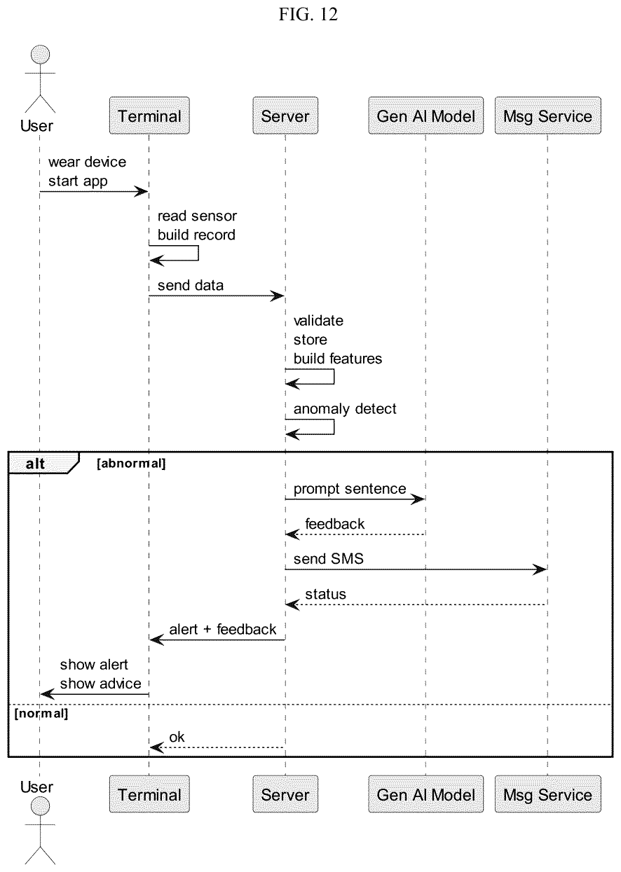

The following describes the processing flow using FIG. 12.

### Step 1:

The terminal establishes a connection with the detection apparatus and acquires raw biological information.

The terminal uses a short-range wireless interface to pair with the detection apparatus and to subscribe to measurement notifications. The terminal receives packets containing raw sensor readings such as heart rate counts and pressure values.

Input: low-level sensor packets including encoded heart rate and blood pressure values.

Processing: the terminal decodes the packets according to a predefined communication protocol, converts raw bytes into numerical values with appropriate units, and attaches metadata such as a timestamp and a user identifier.

Output: structured measurement records containing fields such as user ID, heart rate, systolic pressure, diastolic pressure, and timestamp.

### Step 2:

The terminal transmits the structured measurement records to the server over a communication network.

The terminal formats the structured measurement records into a request body and sends the data via a secure communication protocol to an endpoint exposed by the server.

Input: structured measurement records created in Step 1.

Processing: the terminal serializes the structured records into a transport format, attaches authentication information, and issues a network request to the server.

Output: a network message containing the serialized measurement records delivered to the server.

### Step 3:

The server receives the network message and converts the payload into a table-type data structure.

The server extracts the request body from the network message, parses the transport format, and validates each field for range and format.

Input: serialized measurement records received from the terminal.

Processing: the server deserializes the data, checks that fields such as heart rate and blood pressure fall within plausible bounds, discards or logs invalid records, and inserts valid records as rows into a table-type data structure with predefined columns.

Output: an updated table-type data structure containing validated measurement rows ready for analysis.

### Step 4:

The server constructs feature vectors for anomaly detection from the table-type data structure.

The server selects recent rows associated with a given user, orders them by timestamp, and forms numerical feature arrays.

Input: the updated table-type data structure from Step 3.

Processing: the server extracts columns such as heart rate, systolic pressure, and diastolic pressure; computes derived temporal features such as moving averages, differences between consecutive measurements, and variability indicators; and concatenates these values into multi-dimensional feature vectors.

Output: a batch of feature vectors representing current and recent biological information for the user.

### Step 5:

The server normalizes the feature vectors according to a pre-defined scaling model.

The server applies a scaling transform that was determined during training of the anomaly detection model to align the new data with the model's expected input space.

Input: raw feature vectors produced in Step 4.

Processing: the server subtracts pre-computed means and divides by standard deviations for each feature dimension, or applies another normalization scheme, to obtain normalized feature vectors with consistent ranges.

Output: normalized feature vectors suitable for input to the anomaly detection model.

### Step 6:

The server executes anomaly determination processing using a trained machine learning model.

The server feeds the normalized feature vectors into the anomaly detection model and evaluates whether the current measurements deviate from learned normal patterns.

Input: normalized feature vectors from Step 5.

Processing: the server passes the vectors through the anomaly detection algorithm, which computes anomaly scores based on internal parameters, and compares the scores to one or more thresholds to assign anomaly flags.

Output: anomaly determination results including an anomaly score and a binary anomaly flag for each analyzed measurement set.

### Step 7:

The server categorizes the type and severity of any detected abnormal value.

The server inspects the anomaly determination results and the corresponding measurement values to generate descriptive labels and severity levels.

Input: anomaly flags and scores from Step 6, and the original measurement values associated with those results.

Processing: the server determines the anomaly type (for example, high heart rate or high blood pressure) by comparing measured values with configured normal ranges, and calculates a severity level based on the magnitude of deviation and the anomaly score.

Output: structured anomaly descriptors containing fields such as anomaly type, severity, and associated measurement values.

### Step 8:

The server records the anomaly descriptors and temporal feature values in storage.

The server updates persistent records to maintain an audit trail of abnormal events and their characteristics.

Input: anomaly descriptors from Step 7 and temporal feature values computed in Step 4.

Processing: the server writes a new alert entry into a storage system, linking the anomaly descriptors, temporal features, user identifier, and timestamps into a single alert record.

Output: a stored alert record that can be retrieved for subsequent prompt generation and notification operations.

### Step 9:

The server generates a prompt sentence for a generative AI model based on the alert record.

The server retrieves the alert record and transforms the structured fields into a natural language description according to a configurable template and logic.

Input: the alert record stored in Step 8.

Processing: the server selects key fields such as current heart rate, blood pressure, anomaly type, severity, and recent temporal trends; maps these fields into textual phrases; conditionally includes or omits segments depending on severity and available context; and concatenates the phrases into a coherent prompt sentence.

Output: a prompt sentence that encodes the anomaly, severity, and temporal context for use by the generative AI model.

### Step 10:

The server sends the prompt sentence to the generative AI model and obtains feedback information.

The server calls an interface of the generative AI model and submits the prompt sentence as input to produce an explanatory and instructional response.

Input: the prompt sentence generated in Step 9.

Processing: the server transmits the prompt to the generative AI model, receives a generated text response, and may apply formatting and filtering to split the response into logical steps and to remove extraneous or unsuitable content.

Output: feedback information consisting of natural language instructions and advice aligned with the detected abnormal value.

### Step 11:

The server prepares warning information and notification messages for external recipients.

The server constructs concise messages for third-party contacts and more detailed messages for the user's terminal.

Input: anomaly descriptors from Step 7 and the feedback information from Step 10.

Processing: the server composes a short text message containing essential anomaly details for a third-party contact, composes a detailed alert payload for the terminal including the warning information and feedback information, and associates each message with the relevant communication destinations.

Output: a short text message ready for transmission to a third-party contact, and an alert payload ready for transmission to the terminal.

### Step 12:

The server transmits the short text message to a third-party contact via a messaging service.

The server interfaces with an external messaging infrastructure to deliver the warning to the registered contact.

Input: the short text message produced in Step 11 and the contact information stored in association with the user.

Processing: the server invokes a messaging API, supplies the destination address and message content, and processes the response to confirm acceptance or to handle errors.

Output: a delivered or attempted delivery event for the short text message to the third-party contact.

### Step 13:

The server transmits the alert payload to the terminal and updates the terminal's alert state.

The server uses an application-level protocol or a push notification mechanism to send the alert information to the terminal.

Input: the alert payload prepared in Step 11 and the terminal identification associated with the user.

Processing: the server packages the payload into a response or notification, sends it through the appropriate channel, and updates internal status to reflect that a user-facing alert has been issued.

Output: a delivered alert payload containing warning information and feedback information at the terminal.

### Step 14:

The terminal displays the warning information and feedback information to the user.

The terminal receives the alert payload and renders visual elements on the display device.

Input: the alert payload provided in Step 13.

Processing: the terminal parses the warning text and feedback text, creates user interface elements such as notifications and dialog boxes, and formats the feedback into readable steps or paragraphs.

Output: a visual presentation of the alert and the action guidelines on the terminal's display for the user.

### Step 15:

The user observes the displayed information and provides optional input to the terminal.

The user reads the warning and the generated advice and may acknowledge or request additional details.

Input: the visual output shown by the terminal in Step 14.

Processing: the user performs physical actions such as tapping acknowledgment buttons, requesting more information, or dismissing the alert, and the terminal detects these interactions through its input devices.

Output: user interaction signals that can be sent back to the server for logging or further processing.

### Step 16:

The terminal sends user interaction signals and any follow-up measurements to the server.

The terminal informs the server of the user's response and may continue to transmit new measurement data for re-evaluation.

Input: user interaction signals from Step 15 and new structured measurement records from the detection apparatus.

Processing: the terminal formats the interaction logs and measurement records, sends them to the server as network requests, and thereby closes the loop between user actions and system monitoring.

Output: updated data and interaction logs received by the server, enabling the server to refine monitoring and future prompt sentence generation.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional health monitoring systems that process biological information such as heart rate, sleep duration, and physical activity amount often rely on fixed rule sets and static threshold checks implemented in application logic. In many cases, such systems simply collect measurement data, store the data in a database, and perform basic statistical analysis or simple threshold comparison on a per-sample basis. As a result, these systems frequently generate excessive false positives or overlook context-dependent risks because they do not robustly handle data quality issues such as missing values and outliers, do not construct informative higher-level features over time windows, and do not adapt their feedback content to individual users'historical patterns.

From a computer-technology perspective, existing architectures typically separate low-level data processing from any advanced feedback generation logic, and the interaction with generative AI models, if present at all, is ad hoc. For example, prompt sentences sent to generative AI models are often manually crafted or only loosely coupled to the outputs of machine learning models and data pipelines. This results in inefficient use of computational resources, latency in deriving actionable feedback, and an inability to systematically exploit the rich time-series structure of the biological data. Moreover, conventional systems do not provide a unified processing pipeline in which data preprocessing, feature extraction, state estimation, abnormal event detection, and prompt sentence generation are tightly integrated and automatically orchestrated by a processor.

In particular, conventional systems do not adequately address the technical problem of generating, within a computing environment, structured prompt sentences that encode: (i) preprocessed biological data; (ii) time-window-based feature quantities; (iii) machine-learned mental and physical state indices such as stress and sleep indices; and (iv) user-specific baseline and trend information derived from historical data. Without such structured prompt generation under programmatic control, generative AI models cannot reliably produce precise, context-aware feedback, and system performance in terms of accuracy, responsiveness, and personalization remains limited.

Therefore, there is a need for an improved computer-implemented system and server architecture that: (a) automatically performs robust preprocessing of biological digital data, including complementing missing values and removing abnormal values; (b) extracts time-window-based statistical and time-variation feature quantities; (c) computes mental and physical state indices by using a machine learning model; (d) detects abnormal events based not only on threshold exceedance but also on temporal persistence; and (e) constructs, on the server side under processor control, structured prompt sentences that incorporate both current state indices and historical baseline and trend information, and supplies these prompt sentences to a generative AI model to generate customized feedback. Such a system would constitute a concrete improvement in the way computers process health-related time-series data and interface with generative AI models, thereby improving the technical field of automated health monitoring and feedback generation.

The specific processing by the specific processing unit 290 of the data processing device 12 in Example 2 is realized by the following means.

The present invention provides a server comprising a processor and a memory storing instructions that, when executed by the processor, cause the processor to acquire biological information including at least heart rate, sleep duration, and physical activity amount as digital data from a measurement device via a communication network; to store the digital data in a storage device; to preprocess the digital data by executing a data processing program and a data processing library so as to complement missing values, remove abnormal values, and generate preprocessed digital data; to extract, from the preprocessed digital data, feature quantities for each predetermined time window, the feature quantities including at least statistical quantities and time-variation quantities; to input the feature quantities into a machine learning model and calculate mental and physical state indices including at least a stress index and a sleep index; to compare the mental and physical state indices with preset threshold values, determine presence or absence of an abnormal mental and physical state based on whether a state exceeding the threshold values continues for a predetermined time period, and generate abnormal event information when the abnormal state is detected; to acquire, from the storage device, past biological information histories and past mental and physical state index histories for each user, derive baseline states and trend information from the histories, and construct a prompt sentence that includes at least the feature quantities, the mental and physical state indices, the abnormal event information, and the baseline states and trend information; to provide the constructed prompt sentence as input to a generative AI model and obtain feedback information including an action guideline relating to improvement of a health state of mind and body; and to generate abnormal notification information including the feedback information and transmit the abnormal notification information to a terminal of the user for display. This enables a computer-implemented health monitoring system in which the server performs an integrated sequence of technical operations—data cleansing, feature extraction over time windows, machine-learned state estimation, abnormal event detection with temporal persistence, and structured prompt sentence generation for a generative AI model—thereby improving the accuracy, contextual relevance, and personalization of feedback while efficiently utilizing computational resources and reducing the need for manual prompt design.

The term “biological information” refers to numerical or categorical data representing physical or physiological conditions of a living subject, including at least heart rate, sleep duration, and physical activity amount measured over time.

The term “heart rate” refers to a numerical value indicating the number of heartbeats per unit time, typically expressed in beats per minute, acquired from a measurement device.

The term “sleep duration” refers to a time length indicating a total period during which a subject is in a sleep state within a given observation window.

The term “physical activity amount” refers to a quantitative measure of body movement or exertion over time, including at least step count, movement intensity, or energy expenditure.

The term “measurement device” refers to an electronic apparatus configured to sense biological information of a subject and convert the sensed information into digital data, and includes, for example, a wearable device or a sensor unit.

The term “digital data” refers to biological information that has been converted into a machine-readable numerical or symbolic representation suitable for processing by a computing device.

The term “communication network” refers to a wired or wireless data transmission infrastructure that enables digital communication between a measurement device, a terminal, and an information processing device.

The term “information processing device” refers to a computing apparatus, such as a server or cloud-based computer system, configured to execute programs for storing, preprocessing, analyzing, and transmitting digital data.

The term “storage device” refers to a hardware component or logical resource for non-transitory storage of digital data, including at least semiconductor memory, magnetic storage, optical storage, or database storage.

The term “data processing program” refers to executable instructions that, when run by a processor, cause the processor to perform operations on digital data, including but not limited to reading, writing, transforming, and aggregating the data.

The term “data processing library” refers to a reusable software component or collection of software routines that provide functions for manipulating, cleaning, and transforming digital data, including operations such as interpolation, filtering, and statistical computation.

The term “preprocessing” refers to a sequence of operations applied to raw digital data in order to improve data quality and suitability for subsequent analysis, the operations including at least complementing missing values and removing abnormal values.

The term “missing values” refers to data elements that are absent, undefined, or not recorded in a digital dataset at positions where measurements are expected.

The term “abnormal values” refers to data elements that deviate from expected physiological or statistical ranges and are identified as outliers or artifacts to be removed or corrected during preprocessing.

The term “preprocessed digital data” refers to digital data that has undergone preprocessing operations such as missing value completion and abnormal value removal and is ready for feature extraction and analysis.

The term “feature quantities” refers to derived numerical values computed from preprocessed digital data over one or more time windows, representing characteristics of the data such as central tendency, variability, or temporal change.

The term “predetermined time window” refers to a contiguous time interval of fixed or predefined length, such as seconds, minutes, hours, or days, over which feature quantities are calculated.

The term “statistical quantities” refers to feature quantities that summarize properties of data within a predetermined time window, including at least mean, median, variance, standard deviation, minimum, maximum, or percentile values.

The term “time-variation quantities” refers to feature quantities that characterize changes of data over time, including at least differences, gradients, slopes, or measures of variability between successive time points or windows.

The term “machine learning model” refers to a computational model trained on historical data to infer relationships between input feature quantities and output indices, and configured to output mental and physical state indices when provided with new feature quantities.

The term “mental and physical state indices” refers to numerical scores generated by a machine learning model that represent levels or qualities of mental and physical conditions, including at least a stress index and a sleep index.

The term “stress index” refers to a numerical value computed by a machine learning model that estimates a degree of psychological or physiological stress of a subject based on feature quantities derived from biological information.

The term “sleep index” refers to a numerical value computed by a machine learning model that estimates a degree of sleep quality or adequacy of a subject based on feature quantities derived from biological information.

The term “threshold value” refers to a preset numerical reference level used to judge whether a mental or physical state index indicates a normal state or an abnormal state.

The term “abnormal mental and physical state” refers to a condition in which at least one mental and physical state index exceeds a corresponding threshold value for at least a predetermined time period.

The term “abnormal event information” refers to data describing detection of an abnormal mental and physical state, including at least a type of abnormality, a time of occurrence, relevant indices, and duration of the abnormal state.

The term “biological information histories” refers to a collection of past biological information records for a user, stored over a period of time and used for longitudinal analysis.

The term “mental and physical state index histories” refers to stored sequences of past mental and physical state indices for a user, computed at multiple times and used to analyze patterns and trends.

The term “baseline states” refers to reference values or ranges for mental and physical state indices or biological information that characterize typical or normal conditions for an individual user, derived from historical data.

The term “trend information” refers to information describing temporal patterns or directional changes over time in biological information or mental and physical state indices, such as long-term increases, decreases, or cycles.

The term “prompt sentence” refers to a text sequence formatted as an input instruction or query provided to a generative AI model, the text including structured information such as feature quantities, mental and physical state indices, abnormal event information, baseline states, and trend information.

The term “natural language processing technique” refers to a computational method for generating, interpreting, or transforming human-readable text, used to construct or modify prompt sentences and process textual data.

The term “generative AI model” refers to a machine-implemented model trained on data to generate natural-language text or other content in response to an input prompt sentence.

The term “feedback information” refers to output content generated by a generative AI model in response to a prompt sentence, including at least an action guideline relating to improvement of a health state of mind and body.

The term “action guideline” refers to a recommendation or instruction, expressed in natural language, that suggests concrete actions for a user to take in order to improve or manage mental and physical health.

The term “abnormal notification information” refers to data generated by the information processing device that includes at least abnormal event information and feedback information, and is intended to be provided to a terminal for presentation to a user.

The term “terminal” refers to an end-user computing device capable of communication with the information processing device and presenting information to a user, including, for example, a mobile device, a portable terminal, or a general-purpose computer.

The term “display device” refers to a visual output component of a terminal that presents text, graphics, or other visual information to a user.

The term “processor” refers to one or more hardware processing units configured to execute instructions of a program to perform operations including data acquisition, storage control, preprocessing, feature extraction, model inference, abnormality detection, prompt construction, and communication with external devices and models.

The term “memory” refers to one or more non-transitory computer-readable media configured to store programs and data for use by a processor within the server or information processing device.

In one embodiment, a server cooperates with a terminal and a measurement device to implement the claimed system for monitoring a user's mental and physical state and providing customized feedback generated by a generative AI model. The server includes at least one processor, a memory storing executable programs and model parameters, a storage device implemented as a relational database, and a communication interface connected to a communication network. The terminal includes at least one processor, a memory, a display device, and a wireless communication interface. The measurement device is realized as a wearable sensor apparatus configured to acquire biological information such as heart rate, sleep duration, and physical activity amount, and to transmit such information to the terminal and/or the server.

The server executes a data acquisition module, a preprocessing module, a feature extraction module, a state estimation module, an abnormality detection module, a prompt generation module, and a feedback delivery module. The server may execute these modules using a general-purpose operating system on commercially available server hardware or a virtual machine on a cloud computing platform. In one concrete example, the server uses a Python runtime environment installed on a server-class computer. The server uses a data processing library such as a table-oriented numeric processing library (for example, a library like Pandas) and a scientific computation library (for example, a library like NumPy) to perform numerical operations on digital data. The server uses a machine learning library (for example, a library like Scikit-learn) to execute a trained machine learning model that outputs mental and physical state indices. The server also accesses a generative AI model through a network API, the generative AI model being implemented as a neural network, for example a transformer-based large language model.

The terminal operates an application that receives biological information from the measurement device via a short-range wireless communication interface such as Bluetooth Low Energy. The terminal converts raw sensor data into digital data packets with defined fields such as user identifier, timestamps, heart rate values, sleep durations, and physical activity values. The terminal transmits the digital data packets to the server over a wide-area communication network such as the Internet using a secure protocol such as HTTPS. The terminal may temporarily buffer the data in local storage and send the data in batches to reduce communication overhead.

The server stores the received digital data in a storage device, for example in tables of a relational database management system. The server uses normalized tables in which each row corresponds to a measurement record identified by user identifier, timestamp, and measurement type, and the server maintains indexing on these columns to accelerate subsequent queries. By storing the data in this structured format, the server improves data retrieval efficiency and enables time-series operations to be performed using indexed ranges, thereby reducing computational complexity compared to unstructured storage.

The server then performs preprocessing of the stored digital data by executing the preprocessing module. In one embodiment, the server retrieves raw measurement records for each user over a given time interval. The server uses the data processing library to construct an in-memory table in which rows are aligned by timestamp and columns correspond to measurement types (heart rate, sleep duration, physical activity). The server detects missing values by scanning for null entries or missing timestamps and complements such values by interpolation and aggregation. For example, the server uses linear interpolation along the time axis for short gaps in heart rate data, and uses user-specific historical averages or median values for missing daily sleep duration. The server also detects abnormal values by applying rule-based checks (for example, discarding heart rate values below a physiologically implausible minimum or above a maximum) and statistical checks (for example, computing z-scores over a sliding window and marking values exceeding a threshold as outliers). The server replaces abnormal values with interpolated or imputed values. This preprocessing improves data consistency and removes noise before model inference, which in turn reduces error propagation into later stages, thereby improving overall accuracy and robustness of the system.

The server executes the feature extraction module on the preprocessed data. The server partitions the time-series data into predetermined time windows, such as one-minute, five-minute, or one-hour windows. For each time window, the server computes statistical quantities such as mean, median, standard deviation, minimum, maximum, and quantiles of heart rate and activity measurements. The server further computes time-variation quantities such as first-order differences between consecutive windows, slopes obtained from simple linear regression over a window, and measures of variability such as root mean square of successive differences. These feature quantities are stored in a structured feature table. By computing these time-window-based features, the server transforms raw, irregular biological signals into a compact representation that captures temporal patterns relevant to mental and physical state. This structure allows the server to perform more efficient inference with fewer input dimensions while preserving key temporal information, which improves computational efficiency and predictive performance compared to directly feeding raw time-series data.

The server executes the state estimation module to compute mental and physical state indices from the feature quantities. In one embodiment, the server uses a machine learning model trained offline using historical labeled datasets. The machine learning model may be realized as a gradient boosting decision tree model, a random forest model, or a neural network. In a more advanced embodiment, the machine learning model is an artificial neural network with multiple layers, including at least an input layer that receives normalized feature quantities, one or more hidden layers with non-linear activation functions such as rectified linear units, and an output layer that produces continuous values representing a stress index and a sleep index. The server stores model parameters such as weights and biases in the memory. During inference, the server normalizes input feature quantities based on training statistics (for example, mean and variance) and applies matrix multiplications and activation functions layer by layer to compute output indices. Because the model has been optimized using a loss function such as mean squared error between predicted indices and ground-truth labels, and because model parameters have been updated using a gradient-based optimization algorithm such as stochastic gradient descent or an adaptive optimization algorithm, the model can infer indices that capture non-linear relationships between features and mental and physical state. This use of a trained model allows the server to detect subtle patterns in heart rate variability and activity fluctuations that are not captured by simple threshold-based logic, thereby improving detection accuracy.

During training, the server (or an offline training system) acquires a labeled dataset containing feature quantities and corresponding ground-truth stress and sleep scores. The server initializes model parameters randomly or according to a predefined scheme, computes predictions for a batch of training examples, and computes a loss by applying the loss function to the difference between predictions and labels. The server computes gradients of the loss with respect to model parameters by backpropagation, and updates the parameters by moving them in the direction that reduces the loss. The server may divide the training dataset into training and validation subsets and apply early stopping or regularization techniques to prevent overfitting. The resulting trained model is then deployed to the server for inference.

The server executes the abnormality detection module after computing mental and physical state indices. The server compares the stress index and sleep index with preset threshold values that may be stored in a configuration table in the database. The server does not simply check individual samples; instead, the server considers temporal persistence by analyzing sequences of indices over consecutive time windows. For example, the server may detect an abnormal event when the stress index exceeds a threshold for more than a predetermined number of consecutive windows, or when sleep duration aggregated over a given period falls below a minimum. The server implements such logic by scanning the index time series with a sliding window algorithm or by performing range queries in the database and computing counts of above-threshold occurrences. When an abnormal state is detected, the server generates an abnormal event record containing event type, start time, end time, peak index value, and other context information. This use of time-window persistence reduces spurious alarms caused by transient spikes, thereby improving specificity and reducing false positives.

The server further executes a historical analysis submodule that accesses biological information histories and mental and physical state index histories stored in the storage device. The server computes baseline states for each user by aggregating historical indices over extended periods, such as weeks or months, and computing typical values or distributions for stress and sleep indices. The server also computes trend information such as recent increases or decreases in the indices by applying moving averages, trend filters, or regression over historical data. By maintaining per-user baseline and trend profiles, the server can characterize whether a current abnormal event represents a significant deviation from the user's normal state or a continuation of a long-term pattern. This per-user profiling is not a mere human-like manual review of history but an automated set of computations that restructure and compress historical data into format-optimized baseline and trend features.

The server executes the prompt generation module to construct a prompt sentence to be provided to the generative AI model. The server assembles structured content that includes at least the current feature quantities for a given time window or event, the corresponding mental and physical state indices, the abnormal event information, and the baseline states and trend information for the user. The server then converts these structured data into natural-language segments using a rule-based template engine. For example, the server selects a template sentence pattern and fills in variable fields with numeric values and qualitative descriptions derived from the data. As a concrete example, the server may generate a prompt sentence such as: “You are a health advisor. The user's heart rate has been above 100 beats per minute for 35 consecutive minutes, the predicted stress level is 85 out of 100, and the user slept only 5 hours last night, which is below the user's usual average of 7 hours. The stress level has been increasing over the last 3 days. Please provide concise, practical advice for reducing stress and improving sleep, suitable for a non-medical but health-conscious adult.”

In another example related to sleep shortage, the server may generate a prompt sentence such as:
“Create a brief, practical sleep-improvement plan for a user who has slept only 5 hours per night for the last 3 nights, whereas the user's baseline average sleep time is 7.5 hours. Include advice on bedtime routine, screen use, caffeine, and relaxation techniques. The explanation should fit in about 300 words and be understandable to a layperson.”

The server transmits such prompt sentences to the generative AI model using a network API. The generative AI model is implemented as a neural network with a transformer architecture that processes tokenized text sequences. The model comprises an embedding layer that converts tokens into vectors, multiple self-attention layers that compute contextualized representations, feed-forward layers, and a final output layer that predicts token probabilities. The model has been trained on large text corpora using an unsupervised or semi-supervised objective such as next-token prediction. When the server sends the prompt sentence, the model encodes the prompt and generates a continuation in natural language that constitutes feedback information. The server receives the generated text from the model via the API and post-processes the text, for example to remove extraneous content or to enforce length limits.

The server executes the feedback delivery module to integrate the generated feedback information with abnormal event information and transmit abnormal notification information to the terminal. The server may create a message object that includes a summary of the detected abnormal event, the associated indices, and the generated action guideline. The server sends this message to the terminal using a push notification service or via an application-level messaging protocol. The terminal receives the abnormal notification information and presents it on the display device in a structured layout that separates the factual measurements (e.g., numeric graphs of heart rate and stress index) from the recommended actions (e.g., a bulleted list of advice). The user can then review the information and decide whether to follow the recommended actions.

This configuration yields technical effects that go beyond mere automation of human judgment. The server implements a tightly integrated pipeline that transforms raw, noisy, time-series biological data into feature quantities, indices, event records, and prompt sentences under strict data structures and algorithms. By performing preprocessing such as interpolation and outlier removal using a numeric processing library optimized in native code, the server reduces numerical instability and improves data quality, which directly contributes to more accurate model inference. By computing feature quantities in fixed-length windows and storing them in indexed tables, the server reduces the dimensionality of model input and enables efficient batch inference, thereby improving processing speed and reducing computational load on the processor.

Moreover, by enforcing a standardized structure for prompt sentences that encode both current and historical indicators, the server reduces prompt variability and improves the consistency of outputs from the generative AI model. This structure allows the generative AI model to focus on variations in the encoded metrics rather than on irrelevant differences in prompt phrasing, which improves the reliability and repeatability of feedback in a way that manual, ad hoc prompting cannot achieve. Because the server automatically converts numerical signals into templated textual context, the pipeline achieves systematic coupling between time-series analysis and text generation, which is a specific improvement in computer-implemented interaction between analytic models and generative models.

The system also reduces communication load by transmitting biological measurements from the terminal to the server in aggregated or batched formats and by transmitting only summary indices and advice back to the terminal. The server performs heavy computation centrally, taking advantage of optimized numeric libraries and hardware acceleration if available, while the terminal focuses on user interaction. As a result, the system improves energy efficiency on the terminal and enables practical deployment on resource-constrained portable devices.

In alternative embodiments, the server may employ different machine learning architectures for the state estimation module, such as a recurrent neural network or a one-dimensional convolutional neural network directly applied to time-series samples, provided that the model outputs mental and physical state indices. The server may also use different abnormality detection strategies, such as probabilistic thresholds based on percentile distributions, or anomaly scores computed by autoencoder models. The prompt generation module may use alternative templates or may adapt templates based on user preferences or language settings, while still embedding the same core numerical context into the prompt sentences.

In another embodiment, the terminal may also execute a lightweight local model that performs preliminary estimation of state indices and only sends compressed summary features to the server. The server then refines the estimation and constructs prompt sentences based on both local and server-side computations, thereby reducing bandwidth usage and enabling faster detection for time-critical events. In yet another embodiment, the server may adjust threshold values dynamically based on recent trends in baseline states so that the system can adapt to long-term changes in the user's condition without manual reconfiguration.

In all of these embodiments, the server, the terminal, and the measurement device cooperate to implement concrete data structures, algorithmic steps, and model-based computations that transform specific types of time-series biological data into actionable feedback in a manner that improves processing accuracy, efficiency, and robustness of the underlying computer system.

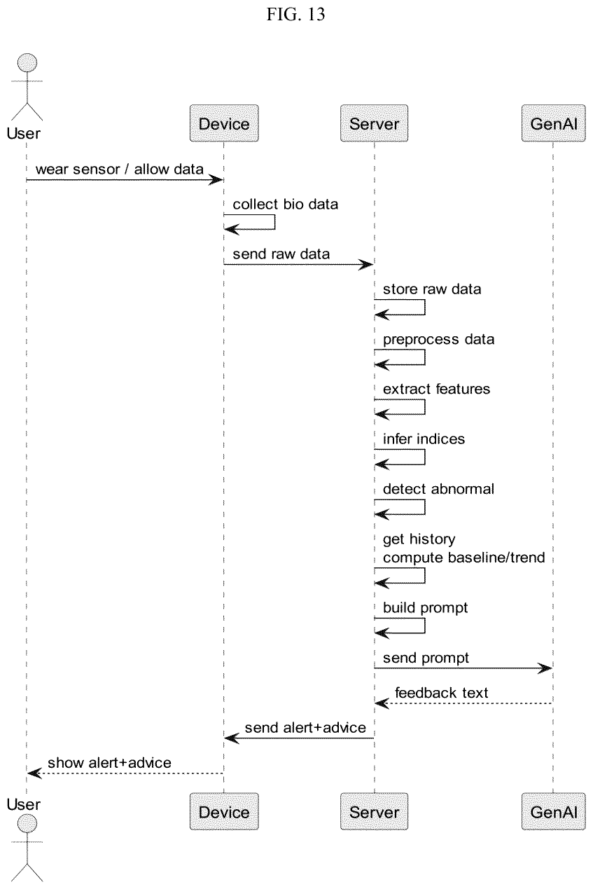

The following describes the processing flow using FIG. 13.

### Step 1:

The user wears a measurement device and authorizes data collection on the terminal.

The input is the user's biological condition in the real world.

The terminal pairs with the measurement device via a wireless interface and receives raw sensor signals such as heart rate pulses, motion signals, and sleep state flags.

The terminal converts the raw signals into digital measurement records by time-stamping each reading and associating it with a user identifier and a measurement type.

The output is a set of structured digital measurement records stored temporarily in the terminal's memory.

### Step 2:

The terminal aggregates and transmits the digital measurement records to the server.

The input is the structured digital measurement records stored in the terminal.

The terminal groups multiple records into a batch, encodes them into a structured message format with fields such as user identifier, timestamp, heart rate value, sleep duration value, and physical activity value, and attaches authentication information.

The terminal sends the batched message to the server over a communication network using a secure protocol.

The output is a network request containing batched digital measurement records delivered to the server.

### Step 3:

The server receives and stores the digital measurement records in a storage device.

The input is the network request containing batched digital measurement records.

The server authenticates the request, parses the structured message, validates field types and ranges, and logs metadata such as reception time and source terminal.

The server inserts each record into one or more database tables, for example a raw measurement table keyed by user identifier and timestamp.

The output is a set of persisted raw measurement records available for later retrieval from the storage device.

### Step 4:

The server performs preprocessing on the raw measurement records.

The input is the set of persisted raw measurement records retrieved from the database for a given user and time range.

The server loads the records into an in-memory tabular structure and aligns them on a regular time grid by resampling and sorting by timestamp.

The server detects missing values by searching for grid positions without measurements and fills them using interpolation or by inserting user-specific historical averages.

The server detects abnormal values by applying rule-based thresholds and statistical outlier detection, and replaces such values with interpolated or imputed values.

The output is a set of preprocessed measurement records in which missing and abnormal values have been corrected to produce consistent time-series data.

### Step 5:

The server extracts feature quantities from the preprocessed measurement records.

The input is the set of preprocessed measurement records aligned on the time grid.

The server divides the time-series data into predetermined time windows, such as fixed-length minute or hour intervals.

The server calculates, for each time window, statistical quantities including mean, median, standard deviation, minimum, maximum, and percentiles for heart rate and physical activity, and aggregated sleep duration.

The server further calculates time-variation quantities such as consecutive differences, slopes, and variability measures across adjacent windows.

The output is a feature dataset in which each record corresponds to a time window and contains multiple feature quantities derived from the preprocessed measurements.

### Step 6:

The server estimates mental and physical state indices using a machine learning model.

The input is the feature dataset for one or more consecutive time windows.

The server normalizes each feature quantity by applying previously determined scaling parameters such as mean and variance.

The server feeds the normalized feature vectors into a trained machine learning model, executes layer-by-layer computations or tree-based evaluations, and obtains continuous output values representing at least a stress index and a sleep index.

The server associates each pair of indices with the corresponding user identifier and time window.

The output is a set of mental and physical state index records, each including at least a stress index and a sleep index for a specific time window.

### Step 7:

The server analyzes the state index records and detects abnormal events.

The input is the set of mental and physical state index records and predefined threshold values for the indices.

The server compares each stress index and sleep index with its corresponding threshold and marks windows as above or below threshold.

The server scans sequences of windows to determine whether an index remains above or below its threshold for at least a predetermined duration, and identifies intervals that satisfy the persistence condition.

The server creates abnormal event records including event type, start time, end time, maximum or minimum index values, and associated user identifier.

The output is a set of abnormal event records representing detected abnormal mental and physical states.

### Step 8:

The server computes baseline states and trend information from historical data.

The input is historical biological information and historical mental and physical state indices for the same user stored in the database.

The server aggregates the historical indices over long periods, calculates typical values such as long-term means and medians, and defines baseline ranges for stress and sleep indices.

The server computes trend information by applying moving averages, regressions, or other time-series analyses to detect increasing or decreasing patterns over recent days or weeks.

The server stores or updates baseline state descriptors and trend descriptors for the user.

The output is a set of baseline and trend descriptors that characterize the user's typical and recent mental and physical state.

### Step 9:

The server generates a prompt sentence for a generative AI model based on current and historical data.

The input is the abnormal event records, the corresponding state index records, the feature quantities for the relevant time windows, and the baseline and trend descriptors for the user.

The server selects an appropriate text template according to the type of abnormal event and fills variable fields with current indices, durations, baseline values, and trend summaries.

The server combines these filled templates into a coherent natural-language description that includes an instruction specifying the role of the generative AI model and the desired style and length of the output.

The output is a prompt sentence in natural language that encodes structured numerical and historical context and is ready to be submitted to the generative AI model.

### Step 10:

The server transmits the prompt sentence to the generative AI model and receives feedback information.

The input is the generated prompt sentence.

The server sends the prompt sentence to a generative AI model through an application programming interface, specifying parameters such as maximum output length and diversity settings.

The generative AI model processes the prompt sentence and generates a textual response that includes an action guideline related to improvement of the user's mental and physical health.

The server receives the textual response and associates it with the corresponding abnormal event record and user identifier.

The output is feedback information in natural language that provides customized recommendations for the detected abnormal event.

### Step 11:

The server constructs abnormal notification information and sends it to the terminal.

The input is the abnormal event records and the associated feedback information.

The server composes a notification payload including a concise summary of the abnormal state, key numerical indicators such as stress index and sleep duration, and the generated action guideline.

The server formats this payload according to a messaging or push notification protocol and transmits it to the terminal identified for the user.

The output is abnormal notification information delivered to the terminal for presentation to the user.

### Step 12:

The terminal presents the abnormal notification information to the user.

The input is the abnormal notification information received from the server.

The terminal parses the notification payload, extracts event details and the action guideline, and renders them on the display device using a user interface layout that may include text, icons, and graphs.

The terminal may allow the user to open a detailed view showing time-series charts of indices and a full version of the feedback text.

The output is a visual display through which the user can recognize the abnormal mental and physical state and read the recommended actions.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional health-monitoring systems typically perform limited numeric threshold checks on biometric signals and then present fixed, template-based messages to users. Such systems suffer from multiple technical drawbacks. First, the processing pipeline on the server side is not configured to integrate heterogeneous data streams, such as time-series biometric data, multimodal emotion inputs (audio, image, and text), and historical user behavior, into a unified machine-interpretable representation. As a result, the generated alerts are either too sensitive or not sensitive enough, leading to frequent false positives and false negatives. Second, conventional servers do not dynamically construct machine-consumable natural-language instructions (prompt sentences) that reflect current analytic context; instead, they rely on static rules or hard-coded messages, which prevents a generative AI model, even if present, from leveraging its full capacity to produce context-appropriate feedback. Third, known systems generally treat a generative AI model as a one-way text generator and do not incorporate user evaluation information in a closed feedback loop to adapt model behavior and prompt construction for individual users over time. This results in feedback that remains generic, repetitive, and poorly aligned with a user's evolving physical and mental condition, thereby reducing user engagement and system effectiveness.

From a computer-technology standpoint, there is no coordinated architecture that: (i) performs server-side statistical analysis of biometric streams together with emotion recognition; (ii) automatically encodes the resulting machine-level analysis into structured prompt sentences; (iii) orchestrates a generative AI model as a back-end component to generate multi-party communications (user feedback and third-party notifications); and (iv) uses explicit user evaluation signals to update prompt construction or model output conditions. Without such an architecture, server resources are inefficiently utilized, the quality and relevance of generated messages cannot be systematically improved, and the system cannot provide robust, adaptive, and scalable health-related feedback. Accordingly, there is a need for an improved computer-implemented system that technically enhances server-side processing by integrating analytical modules, prompt-generation logic, generative AI inference, and feedback-driven adaptation into a unified processing pipeline.

The specific processing by the specific processing unit 290 of the data processing device 12 in Application Example 2 is realized by the following means.

The present invention provides a server comprising a processor configured to store, in a storage device, digital data converted from biological information acquired via a detection device, to analyze the digital data by a statistical processing method to generate a biological state indicator and an abnormality determination result, to identify an emotional state by applying an emotion recognition model to audio information, image information, and character information, to integrate the biological state indicator and the emotional state to calculate a risk level of a physical and mental state and determine necessity of issuing a warning based on the risk level, to generate a prompt sentence including a state description based on the biological state indicator, the emotional state, and the necessity of issuing the warning, to input the prompt sentence into a generative artificial intelligence model so as to cause the generative artificial intelligence model to generate feedback information for a user terminal and notification text for an external contact, to transmit the feedback information to the user terminal and transmit the notification text to the external contact, and to obtain evaluation information from a user regarding the feedback information and update at least one of the prompt sentence and output conditions of the generative artificial intelligence model based on the evaluation information. This enables the server-side computer system to technically improve processing of heterogeneous health-related data by automatically encoding analytic results into machine-interpretable prompt sentences, by orchestrating a generative AI model as a controlled back-end component for generating adaptive multi-party communications, and by closing the loop with user evaluation signals so that subsequent prompts and model outputs are computationally optimized for each user, thereby enhancing detection accuracy, message relevance, and overall efficiency of the health-monitoring platform.

The term “processor” refers to a hardware computing element or a combination of hardware and software components that executes machine-readable instructions to perform data acquisition, analysis, prompt generation, communication control, and model interaction in the system.

The term “detection device” refers to a sensing apparatus configured to measure biological information from a user, such as a wearable sensor, mobile device sensor, or other bio-signal acquisition unit, and to output corresponding electrical or digital signals.

The term “biological information” refers to data representing a physiological or physical state of a user, including but not limited to heart rate, blood pressure, body temperature, activity amount, and sleep-related metrics.

The term “digital data” refers to biological information or other signals that have been converted into a machine-readable numerical or symbolic representation suitable for processing by the processor and storage in a storage device.

The term “communication network” refers to a wired or wireless data-communication infrastructure, including local networks and wide-area networks, through which the detection device, user terminal, and information processing apparatus exchange digital data.

The term “information processing apparatus” refers to a computing system, such as a server or cloud-based computing environment, that is configured to store, analyze, and further process digital data received from external devices.

The term “storage device” refers to a physical or logical data-storage resource, such as a memory unit or database system, in which digital data, analysis results, prompt sentences, and generated text are persistently or temporarily stored.

The term “statistical processing method” refers to a computational technique that applies statistical operations, including aggregation, averaging, variance or deviation calculation, trend analysis, or threshold comparison, to digital data in order to extract patterns or indicators.

The term “biological state indicator” refers to a derived metric or set of metrics obtained from digital data that numerically or categorically represents a current or historical biological condition of the user.

The term “abnormality determination result” refers to information indicating whether a deviation of the biological state indicator from predefined or learned reference values satisfies a condition for being treated as abnormal.

The term “audio information” refers to recorded or streamed sound signals associated with a user, including spoken utterances and non-speech acoustic cues, that are processed to infer emotional or contextual characteristics.

The term “image information” refers to still image data or image frames obtained from a camera or equivalent visual sensor, including facial images or body images that are analyzed to estimate a user's emotional state.

The term “character information” refers to text data associated with a user, including manually entered text, transcribed speech, or messages obtained from communication services, which are processed for emotion or sentiment analysis.

The term “emotion recognition processing apparatus” refers to a function or module of the information processing apparatus that applies one or more models to audio information, image information, and character information in order to identify an emotional state.

The term “emotion recognition model” refers to a computational model, such as a machine-learning or pattern-recognition model, trained to classify or estimate emotional states from one or more input modalities.

The term “emotional state” refers to a classification or score representing a user's psychological or affective condition, such as stress, anxiety, calmness, or similar states, as identified by the emotion recognition model.

The term “risk level of a physical and mental state” refers to a numerical or categorical evaluation that reflects a degree of potential concern regarding the user's combined physical and emotional condition, derived from the biological state indicator and the emotional state.

The term “necessity of issuing a warning” refers to a determination result indicating whether the risk level of the physical and mental state satisfies a condition that requires generation and transmission of a warning or alert message.

The term “prompt sentence” refers to a machine-consumable natural-language expression that encodes a state description, including at least the biological state indicator, the emotional state, and the necessity of issuing a warning, and that is supplied as input to a generative artificial intelligence model.

The term “state description” refers to a natural-language or structured representation summarizing relevant analytical results, including biological, emotional, and risk-related information about the user, for use in the prompt sentence.

The term “generative artificial intelligence model” refers to a computational model that receives a prompt sentence as input and generates natural-language text, including feedback information or notification text, based on learned patterns.

The term “feedback information” refers to natural-language content generated by the generative artificial intelligence model and intended for presentation to the user, including advice, explanations, or action guidelines related to the user's physical and mental state.

The term “notification text” refers to natural-language content generated by the generative artificial intelligence model and intended for transmission to an external contact, summarizing the user's condition or risk level and optionally suggesting follow-up actions.

The term “user terminal” refers to an end-user device, such as a mobile communication device, wearable device, or personal computing device, that is configured to receive and display feedback information and to transmit user input or evaluation information to the server.

The term “external contact” refers to an entity other than the user, including an individual or an organization, that is designated to receive notification text about the user's physical and mental state.

The term “evaluation information” refers to data provided by the user that expresses a response to the feedback information, including ratings, selections, or comments indicating usefulness, appropriateness, or satisfaction.

The term “output conditions of the generative artificial intelligence model” refers to configuration parameters or control settings, such as style, length, or level of detail of generated text, that influence the manner in which the generative artificial intelligence model produces feedback information or notification text.

The term “constituent elements of the prompt sentence” refers to component segments, fields, or pieces of information that are included in the prompt sentence, such as summaries of biological indicators, emotional states, risk levels, and instruction directives.

The term “weighting of the prompt sentence” refers to relative emphasis or importance assigned to different constituent elements within the prompt sentence, which affects how the generative artificial intelligence model interprets and utilizes those elements when generating output.

The term “history information of the user” refers to previously stored data associated with the user, including past biological information, past emotional states, prior alerts, and prior evaluation information, that can be referenced when generating new feedback information.

In one embodiment, a server, a terminal, and a detection device cooperate to implement the claimed system. The server provides an information processing apparatus, the terminal provides a user terminal, and the detection device provides one or more sensors that acquire biological information from a user.

The terminal uses hardware sensors such as an optical heart-rate sensor, an accelerometer, a temperature sensor, and a blood-pressure sensor, which may be integrated into a wearable device or a mobile communication device. The terminal converts raw electrical signals from the sensors into digital data using an analog-to-digital converter and a device-level driver. The terminal associates each measurement with metadata including a user identifier, a device identifier, a timestamp, and a sensor type identifier, and encapsulates these values in a structured data record, such as a row in a logical table or an object with named fields.

The terminal transmits the structured data records to the server via a communication network. The terminal establishes a secure channel using a standard transport protocol, such as a secure hypertext transfer protocol, and sends the data in batches to reduce communication overhead. The terminal can buffer data in a local storage area and adaptively determine a transmission interval based on a sampling rate and network conditions. By batching and compressing multiple measurements, the terminal reduces the number of network round-trips and decreases bandwidth consumption, thereby lowering power consumption on the battery-powered terminal.

The server executes software modules implemented on a general-purpose computing platform. The server includes a storage device implemented by a relational data management system, such as a relational database engine, and a memory subsystem used for in-memory processing by application logic. The server stores incoming digital data in normalized tables, for example a biometric table with columns for user identifier, timestamp, sensor type, and value, and an emotion table with columns for modality type and derived emotion labels. The server creates indexes on user identifier and timestamp to support efficient time-window queries, which directly improves query latency for subsequent analysis.

The server uses an analysis module implemented in a programming environment such as a general-purpose interpreted language. The analysis module loads relevant data from the storage device into in-memory structures, such as two-dimensional labeled arrays, and performs statistical processing. The server calculates measures such as rolling means, medians, standard deviations, and percentile thresholds over configurable windows of time. The server also computes user-specific baselines by aggregating historical data, for example by calculating an average resting heart rate over multiple days when an activity level is below a certain threshold. The server derives a biological state indicator by comparing current values to these baselines and by assigning normalized deviation scores.

The server determines an abnormality determination result by applying rule sets and learned thresholds to the biological state indicator. For example, the server determines that a heart-rate indicator is abnormal when a standardized deviation exceeds a predetermined value for a continuous period. The server encodes such conditions as explicit decision logic, so that each abnormality flag is traceable to numeric criteria. This explicit logic reduces ambiguity and improves reproducibility of the abnormality determination.

The server further processes multimodal input to identify an emotional state. The terminal acquires audio information via a microphone and image information via a camera and transmits sampled frames and audio segments to the server. In some embodiments, the terminal locally performs initial compression or feature extraction to reduce bandwidth, for example by converting raw audio to a lower-bit-rate encoded stream and by resizing image frames. The terminal can also transmit character information obtained from user input fields or from external message services.

The server uses an emotion recognition processing apparatus realized in software modules that implement image processing and signal processing. The server uses a computer vision library to detect a face within image frames and to locate key landmarks. The server computes features such as distances and angles between facial landmarks and feeds them into an emotion recognition model. The emotion recognition model can be a convolutional neural network that includes multiple convolutional layers, pooling layers, and fully connected layers trained on labeled emotion datasets. The model outputs probability scores for multiple emotion categories, such as stress, calm, or neutral.

The server processes audio information by extracting features such as mel-frequency cepstral coefficients, pitch contours, and energy, and inputs these features into a neural network model, such as a recurrent architecture or a transformer-based encoder, trained to classify vocal emotional states. The server processes character information by tokenizing text and applying a transformer-based classifier trained for sentiment and emotion detection. The server then aggregates the outputs of these modality-specific models, for example by computing a weighted average of probability vectors, to obtain a unified emotional state for each time period.

The server integrates the biological state indicator with the emotional state to compute a risk level of a physical and mental state. The server uses a rule-based fusion layer or a small neural network that takes as input numeric features such as standardized deviations of biological indicators, emotion scores, and temporal features (e.g., duration of elevated stress). The fusion layer produces a scalar risk level or a discrete risk category. In some embodiments, the fusion layer is a feed-forward network trained using supervised learning with labels representing expert-designated risk conditions. The server uses an objective function, such as cross-entropy loss, during training and updates weights by gradient-based optimization. By training the fusion layer on labeled combinations of signals, the server reduces false positives and false negatives compared to using independent thresholds on each individual signal.

The server determines a necessity of issuing a warning based on the risk level. The server compares the risk level to one or more thresholds that may depend on user history or configuration. The server records the risk level and the necessity flag in the storage device as part of an alert record, thereby creating an auditable history of decision outcomes.

The server generates a prompt sentence that encodes a state description. The state description includes at least the biological state indicator, the emotional state, the risk level, and the necessity of issuing a warning, expressed in natural language but following a structured pattern. The server constructs the prompt sentence by filling predefined template slots with data, for example: a section describing numerical values, a section describing detected emotions, a section describing context such as duration and recent trends, and explicit instructions regarding required outputs.

In one example, the server generates a prompt sentence such as:
“The user's heart rate is 110 bpm, which is significantly higher than their normal resting range of 70-80 bpm, and the emotion analysis indicates high stress for the last 15 minutes. Generate a short alert message for the user and specific guidance for immediate relaxation.”

In another example, the server generates a prompt sentence such as:
“The user has repeatedly posted messages indicating work-related stress, and their average sleep duration over the past three days is less than 5 hours. Generate an alert message and three concrete recommendations to reduce stress and improve sleep.”

In still another example, the server generates a prompt sentence such as: “The user's heart rate has exceeded 100 bpm for 20 minutes and the emotion analysis indicates high stress. Write a concise notification for the user's emergency contact explaining the situation and suggesting that they check on the user.”

The server inputs the prompt sentence to a generative AI model. The generative AI model is implemented as a neural network architecture, such as a transformer-based language model with multiple attention layers, feed-forward layers, and positional encodings. The server provides the prompt sentence to the model through an inference interface, which may be executed locally on the server or on a remote computing node. The model internally represents tokens using embedding vectors and processes them with multi-head self-attention, allowing the model to focus on different parts of the prompt sentence, such as numeric values or explicit instruction phrases.

The server configures model parameters including maximum generated length, sampling temperature, and beam width to control output style. The server specifies that the model output should be logically separated into at least one user-facing feedback segment and optionally one third-party notification segment. The server can embed delimiters or role tags within the prompt sentence to enforce this structure. This explicit structuring of the prompt sentence and configuration of generation parameters causes the generative AI model to behave as a controllable component within the server pipeline, rather than a free-form text generator.

The server receives generated text from the generative AI model and parses the output. The server splits the output into segments according to pre-agreed delimiters or markers and classifies each segment as feedback information or notification text. The server optionally applies filters to remove inappropriate content or to enforce domain-specific constraints, such as prohibiting certain medical instructions. By controlling model parameters and post-processing, the server ensures that the generative AI model behaves reliably and predictably in a technical system context.

The server transmits the feedback information to the user terminal. The server encapsulates the feedback information together with metadata such as alert identifiers and risk levels into a compact message format and sends it over the communication network. The user terminal decodes the message and updates its user interface. The terminal displays a summary alert and the detailed guidance text in a structured layout. The terminal may use local text-to-speech processing to present the feedback audibly. This interaction causes physical changes on display hardware and speakers, linking the abstract processing to tangible device control.

The server transmits notification text to an external contact, such as a caregiver or supervisor, when the necessity of issuing a warning satisfies configured conditions. The server uses a messaging subsystem that can send electronic messages via different channels, for example an electronic mail protocol or a text message service. The server records each sent notification in the storage device, including timestamps and delivery status. This operation controls networking hardware and external communication infrastructure and results in changes to remote devices operated by the external contact.

The user interacts with the terminal to provide evaluation information regarding the feedback information. The user may rate the usefulness of the advice or indicate whether the alert was appropriate. The terminal sends the evaluation information to the server. The server stores evaluation records, associating each record with the corresponding prompt sentence and model output. The server uses this data for adaptation.

The server updates at least one of the prompt sentence structure and the output conditions of the generative AI model based on evaluation information. For example, the server may adjust the weights applied to different elements of the state description, increase or decrease emphasis on certain features such as emotional history, or change the length and level of detail of generated text for particular users. The server can maintain user-specific configuration profiles that record preferred guidance styles. The server can also retrain or fine-tune internal control modules, such as a smaller model that predicts which prompt template will yield better feedback quality. By incorporating evaluation information into the control logic, the server enhances personalization and improves the objective performance of the system over time, measured by reduced complaint rates or improved adherence.

From a technical perspective, this architecture improves several aspects of computer technology. By using indexed storage structures and windowed statistical processing on a server, the system reduces the amount of data that must be scanned for each analysis, thereby improving processing speed and scalability. By computing user-specific baselines and standardized deviation scores, the server increases detection accuracy and reduces false alerts compared with simple global thresholds. By structuring prompt sentences with explicit sections and control tokens, the system improves the determinism and consistency of generative AI outputs and reduces the need for manual post-editing.

The system also improves communication efficiency. The terminal batches and compresses data to reduce uplink load, while the server computes derived features so that downstream modules, including the generative AI model, operate on compact representations instead of raw signals. This reduces unnecessary data transfer and memory usage. The integration of multimodal emotion recognition and biometric analysis in a single fusion layer reduces repeated passes over the data and allows reuse of intermediate features, which decreases computational overhead.

The generative AI model is used in a technically constrained manner that differs from human manual drafting of messages. The server automatically encodes internal numeric and categorical analysis into prompt sentences that contain structured, machine-designed patterns, including explicit delimiters, instruction tokens, and normalized descriptions. This encoding is not a direct translation of human verbal reasoning but a systematically designed representation optimized for consumption by a neural language model. As a result, the model can generate more consistent, machine-interpretable segments that the server can further process and log. The closed feedback loop, in which the server updates prompt structures and output conditions based on evaluation information, creates an adaptive control system that adjusts behavior in ways that are not feasible with static human-written templates.

Alternative embodiments can vary specific implementations while remaining within the same inventive concept. In one embodiment, the emotion recognition model is implemented as a recurrent neural network rather than a convolutional network. In another embodiment, the generative AI model is a sequence-to-sequence transformer with encoder-decoder architecture rather than a decoder-only transformer, and the prompt sentence is split into source and control segments. In another embodiment, the storage device uses a column-oriented database to further optimize analytical queries. In still another embodiment, the fusion layer is implemented as a gradient-boosted decision tree model rather than a neural network, to provide explainable intermediate decision boundaries.

In each embodiment, the server, the terminal, and the detection device cooperate through defined data structures, statistical modules, emotion recognition models, generative AI components, and feedback-driven adaptation mechanisms. This cooperation enables the system to process biological information and emotional inputs in a technically advanced way, improving detection accuracy, computational efficiency, and communication control beyond conventional rule-based or template-based systems.

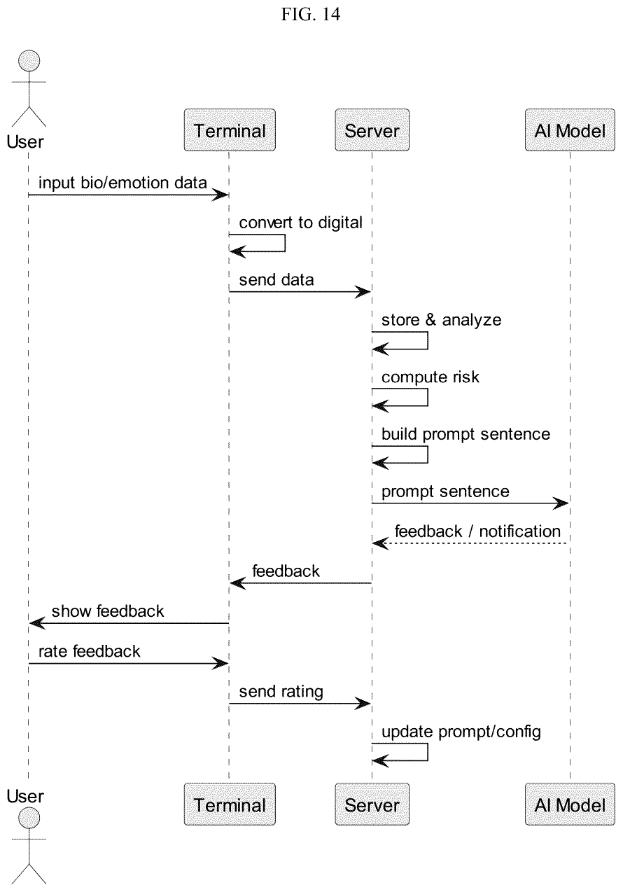

The following describes the processing flow using FIG. 14.

### Step 1:

Terminal acquires biological information from sensors and converts it into digital data.

Terminal receives as input raw electrical signals from a detection device, such as an optical heart-rate sensor, an accelerometer, a temperature sensor, and a blood-pressure sensor worn or carried by the user. Terminal performs analog-to-digital conversion on these signals, associates each measurement with a timestamp, a user identifier, and a sensor type identifier, and generates as output structured digital records containing fields such as {user_id, device_id, timestamp, sensor_type, value}. Terminal additionally acquires optional contextual inputs such as user-entered stress ratings and short text comments through a graphical user interface and embeds these values into the same structured records.

### Step 2:

Terminal preprocesses and transmits digital data to the server via a communication network.

Terminal takes as input the structured digital records generated in Step 1 and performs data preprocessing, including unit normalization (for example, converting milliseconds to seconds), simple filtering of obvious outliers (for example, discarding impossible values), and aggregation of high-frequency readings into time windows (for example, computing 60-second averages). Terminal then compresses a batch of records into a message payload, establishes a secure session using a secure hypertext transfer protocol, and transmits the compressed payload to the server. The output of this step is an encrypted network message containing multiple preprocessed digital records.

### Step 3:

Server receives digital data and stores it in a storage device.

Server receives as input the encrypted network message from the terminal, decrypts the payload, validates the schema of each record, and rejects records that lack required fields or exceed configured limits. Server then maps valid records into database rows and executes insert operations into a biometric data table that includes columns for user identifier, timestamp, sensor type, and value. Server also creates or updates index entries for user identifier and timestamp to speed up subsequent queries. The output of this step is a set of persistent database records that represent time-stamped biological information ready for analysis.

### Step 4:

Server retrieves a time window of biological data and computes biological state indicators.

Server takes as input a user identifier and a target analysis time period, queries the database for all biometric records for that user within the time window, and loads the query result into an in-memory structure such as a two-dimensional labeled array. Server then performs data processing operations including sorting by timestamp, filling missing values, and computing functions such as rolling means, standard deviations, and percentile values for each sensor type. Server compares current measurements to historical baselines stored in the database, computes normalized deviation scores, and aggregates these scores into biological state indicators (for example, “elevated heart rate,” “reduced activity”). The output of this step is a structured object that contains per-metric indicators and deviation values for the given time window.

### Step 5:

Terminal acquires multimodal emotion-related data and sends it to the server.

Terminal receives as input audio signals from a microphone and image frames from a camera during user interaction or at configured intervals. Terminal digitizes and optionally downsamples these signals, encodes the audio into a compressed format, and resizes image frames to reduce resolution while preserving facial regions. Terminal associates the digitized signals with timestamps and user identifiers and packages them into an emotion payload. Terminal then transmits this payload to the server over the secure communication channel. The output of this step is a network message containing audio samples and image frames linked to the same time axis as the biological information.

### Step 6:

Server performs emotion recognition on audio, image, and character information.

Server receives as input the multimodal emotion payload containing audio and image data, and optionally receives character information such as user-entered text or externally obtained posts. Server first applies a face detection algorithm to each image frame to crop and normalize facial regions; server then computes facial features, such as distances between landmarks, and feeds these features into a convolutional neural network trained for emotion classification. In parallel, server extracts acoustic features such as mel-frequency cepstral coefficients and pitch contours from the audio, constructs feature vectors, and feeds them into a neural network model trained for vocal emotion recognition. Server also tokenizes character strings and inputs token sequences into a transformer-based classifier for sentiment and emotion detection. Server combines these modality-specific outputs using weighted averaging or another fusion strategy to identify a dominant emotional state and confidence scores. The output of this step is an emotional state object that includes labels such as “high stress” or “calm” and associated probabilities.

### Step 7:

Server integrates biological state indicators and emotional state to compute a risk level and determine warning necessity.

Server takes as input the biological state indicator object from Step 4 and the emotional state object from Step 6. Server constructs a feature vector that includes normalized biological deviations, emotion probabilities, and temporal attributes such as duration of abnormal readings. Server applies a fusion function, which may be a rule-based logic module or a feed-forward neural network, to map this feature vector to a scalar risk level and a discrete risk category. Server then compares the risk level to one or more configurable thresholds and determines a necessity of issuing a warning as a Boolean or categorical result. The output of this step is a risk assessment object containing the computed risk level, risk category, and warning-necessity flag.

### Step 8:

Server generates a prompt sentence that encodes a state description for a generative AI model.

Server receives as input the biological state indicators, the emotional state, and the risk assessment object. Server selects a prompt template according to the type of communication to be generated (for example, user advice or third-party notification) and fills template slots with concrete values, including numerical measurements, baseline ranges, emotion labels, and risk categories. Server concatenates these filled segments with instruction phrases that specify the desired output structure. For example, server may generate a prompt sentence such as: “The user's heart rate is 110 bpm, which is significantly higher than their normal resting range of 70-80 bpm, and the emotion analysis indicates high stress for the last 15 minutes. Generate a short alert message for the user and specific guidance for immediate relaxation.” The output of this step is a text string representing a prompt sentence that fully describes the current state in a machine-oriented natural-language format.

### Step 9:

Server inputs the prompt sentence to a generative AI model and obtains feedback information and notification text.

Server takes as input the prompt sentence from Step 8 and passes it to a generative AI model implemented as a neural language model, such as a transformer architecture running on the server or on a connected computing resource. Server specifies generation parameters including maximum output length, sampling temperature, and formatting constraints. The generative AI model tokenizes the prompt sentence, processes token embeddings through multiple attention and feed-forward layers, and generates output tokens that form natural-language text segments. Server decodes these tokens into text and, if the output contains markers indicating multiple segments, splits the text into a user-facing feedback segment and a third-party notification segment. The output of this step is at least one feedback information string for the user and optionally one notification text string for an external contact.

### Step 10:

Server validates, stores, and distributes feedback information to the user terminal.

Server receives as input the feedback information text generated in Step 9. Server applies rule-based filters to check for prohibited phrases or missing required elements, and truncates or reformats the text to conform to user interface limits. Server stores the validated feedback information along with the originating prompt sentence and risk assessment in a feedback table in the database. Server then constructs a response message containing alert identifiers, risk levels, and the feedback text, and transmits this message to the terminal over the secure network connection. Terminal receives this message as input, parses the content, and updates the user interface to display the alert headline and detailed guidance, optionally triggering text-to-speech output. The output of this step is a visual and/or audible presentation of tailored feedback on the user terminal.

### Step 11:

Server validates, stores, and transmits notification text to an external contact when appropriate.

Server takes as input the notification text string from Step 9 together with the warning-necessity flag and user notification preferences. Server determines whether external notification is required based on the risk level, the warning-necessity flag, and stored contact settings. When required, server encapsulates the notification text, user identifier, and time information into a message formatted for an external communication channel, such as an electronic mail protocol or a short message service. Server sends the formatted message through a communication gateway and records a notification log entry in the database. The output of this step is a delivered notification message presented on a device of the external contact.

### Step 12:

User provides evaluation information regarding feedback, and terminal sends the evaluation to the server.

User views the feedback information on the terminal and, as input to this step, selects evaluation options such as “helpful,” “not helpful,” or specific satisfaction levels, and may optionally enter free-form comments. Terminal converts this user interaction into structured evaluation data, including fields such as alert identifier, rating, and comment text. Terminal transmits this evaluation payload to the server over the secure communication channel. The output of this step is a set of evaluation records available for adaptive processing on the server.

### Step 13:

Server updates prompt construction and generative AI output conditions based on evaluation information.

Server receives as input the evaluation records from Step 12 and the stored association between each evaluation record and its corresponding prompt sentence and generated feedback. Server analyzes evaluation data by aggregating ratings per template type, per user, and per content pattern, and computes statistics such as average usefulness scores and variance. Server then adjusts configuration parameters, for example by increasing the weight of certain state elements in prompt templates, changing instructions to request shorter or more detailed outputs, or modifying generation parameters such as temperature for particular users. Server may also update a lightweight control model that predicts which prompt sentence variant should be used for a given combination of biological and emotional states. The output of this step is an updated set of prompt templates and model configuration parameters that will be used in subsequent executions, thereby closing the adaptation loop and improving the relevance and effectiveness of future feedback.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

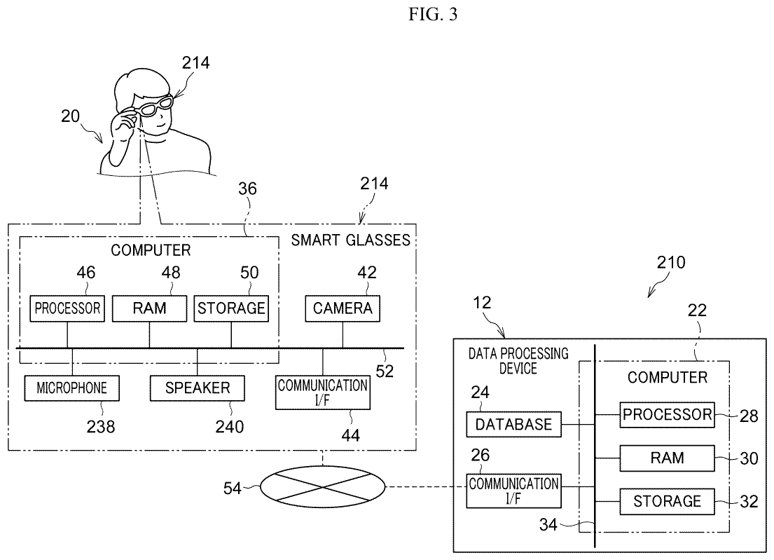

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

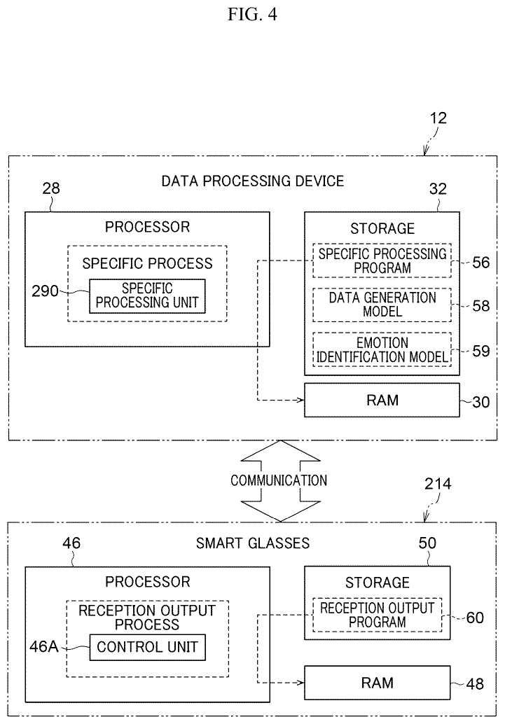

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

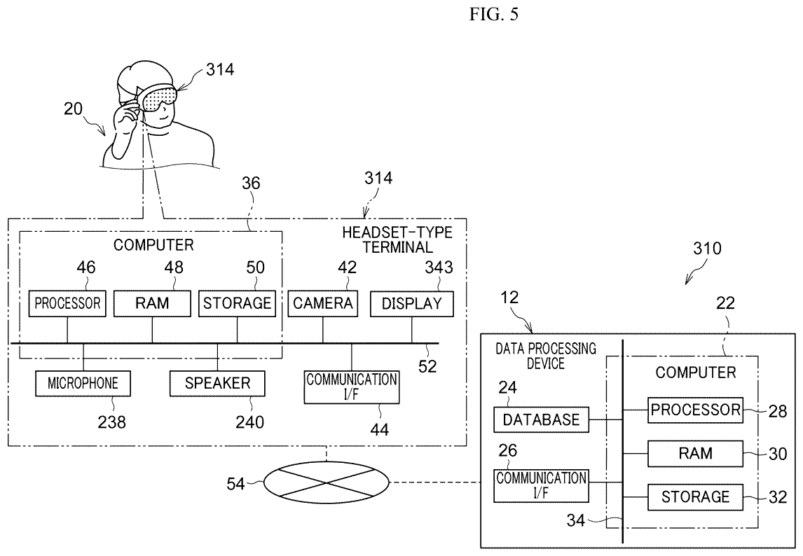

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

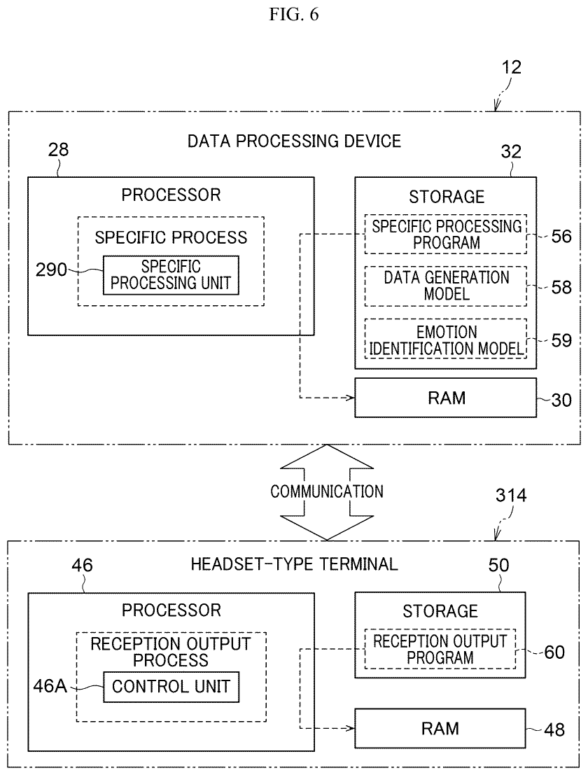

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

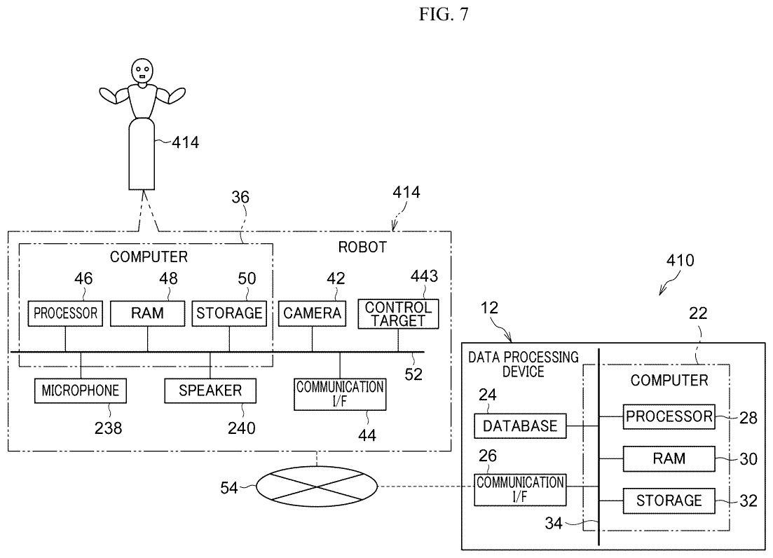

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

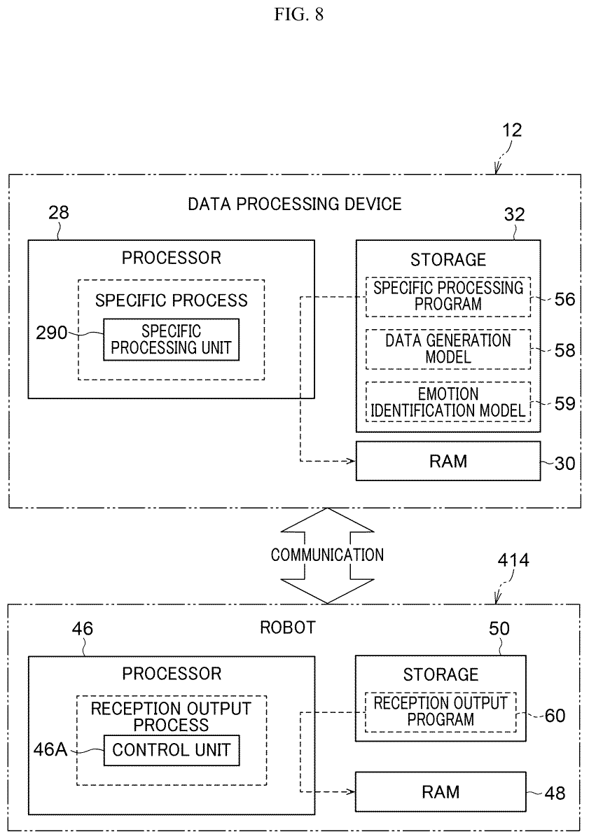

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

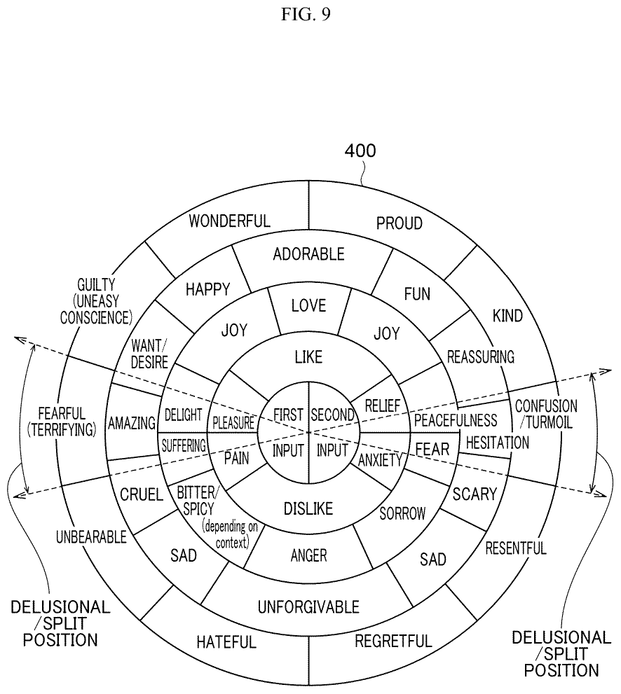

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

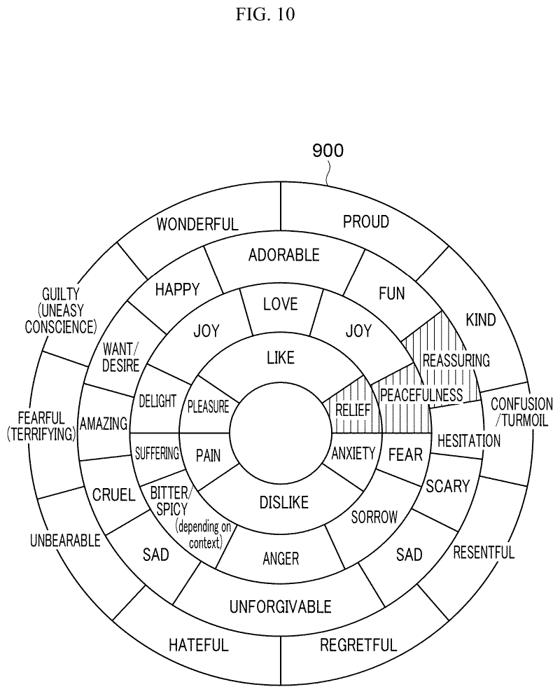

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire biological information from a detection apparatus and convert the biological information into digital data,
transmit the digital data to an information processing apparatus via a communication network in an encrypted manner, and store the digital data as structured data in a storage device of the information processing apparatus,
obtain the digital data stored in the information processing apparatus and historical data stored in the past, and execute statistical analysis processing including calculation of statistical values, time-series analysis, and anomaly detection by numerical computation so as to generate health state evaluation data for each user,
construct input information including the health state evaluation data and user attribute information as a prompt sentence that instructs a generative artificial intelligence model to generate a natural-language response, and transmit the prompt sentence to the generative artificial intelligence model, and
convert natural-language feedback information obtained from the generative artificial intelligence model into output data displayable on a display unit of a user terminal, transmit the output data to the user terminal, and store, in the storage device, a history indicating a correspondence among the feedback information, the prompt sentence, and the health state evaluation data.

### (Supplementary 2)

The system according to supplementary 1,
wherein the processor is configured to cause the generative artificial intelligence model to perform natural-language processing based on the health state evaluation data and the historical data included in the prompt sentence, and to generate evaluation content and behavioral proposals that differ for each physical and mental state of the user, and wherein the feedback information includes evaluation results and corresponding behavioral guidelines for a plurality of items relating to sleep, physical activity, dietary habits, and psychological load.

### (Supplementary 3)

The system according to supplementary 1,
wherein the processor is configured to, in the statistical analysis processing on the historical data, calculate at least an average value, a variability index, and a trend index over a predetermined period, identify, based on the trend index, at least a change trend of sleep duration, a variation trend of circulatory system indicators, and a variation trend of psychological load indicators, and include these trend pieces of information in the prompt sentence so that the generative artificial intelligence model generates customized feedback information that takes into account temporal changes for each user.

### Application Example 1

### (Supplementary 1)

A system comprising a processor,
wherein the processor is configured to
acquire biological information from a detection apparatus that measures the biological information and converts the biological information into digital data,
transmit the digital data to an information processing apparatus via a communication network,
store the digital data, in the information processing apparatus, in a table-type data structure held in a storage unit, and execute anomaly determination processing using a machine learning processing unit to determine presence or absence of an abnormal value based on the digital data,
when the abnormal value is detected, transmit, by a notification processing unit, a short text message including warning information to a third-party contact,
when the abnormal value is detected, cause a portable information terminal having a display device to display the warning information relating to the abnormal value,
generate a prompt sentence to be input to a generative information processing model, based on the abnormal value and history information of the biological information, and
cause the generative information processing model to analyze the prompt sentence and generate feedback information including an action guideline corresponding to the abnormal value, and cause the portable information terminal to display the feedback information.

### (Supplementary 2)

The system according to supplementary 1,
wherein the processor is configured to dynamically generate the prompt sentence such that a type of the abnormal value, a severity of the abnormal value, and feature values indicating temporal changes of the biological information are included in the prompt sentence based on a result of the anomaly determination processing by the machine learning processing unit in the information processing apparatus, and to format the prompt sentence in a form analyzable by the generative information processing model using natural language processing technology.

### (Supplementary 3)

The system according to supplementary 1,
wherein the processor is configured to cause the generative information processing model to generate, as the feedback information, information including stepwise action guidelines for improvement of a physical and mental condition and advice regarding necessity of contacting a medical institution or another organization when the abnormal value continues or worsens, based on the history information of the biological information, the result of the anomaly determination processing, and context information including user attribute information.

### Example 2

### (Supplementary 1)

A system comprising a processor,
wherein the processor is configured to
acquire biological information including at least heart rate, sleep duration, and physical activity amount as digital data by using a measurement device that measures biological information; and
transmit the digital data to an information processing device via a communication network; and
cause the information processing device to store the digital data in a storage device and to perform preprocessing on the digital data by using a data processing program and a data processing library, the preprocessing including at least complementing missing values and removing abnormal values; and
cause the information processing device to calculate, by using a machine learning model, mental and physical state indices including at least a stress index and a sleep index as numerical values from the preprocessed digital data; and
cause the information processing device to compare the mental and physical state indices with preset threshold values, to determine presence or absence of an abnormal mental and physical state based on whether a state exceeding the threshold values continues for a predetermined time period, and to generate abnormal event information relating to occurrence of the abnormal state; and
cause the information processing device to construct a prompt sentence based on the abnormal event information and the mental and physical state indices by using a natural language processing technique, and to generate, as input to a generative AI model, the prompt sentence to be transmitted to the generative AI model; and
cause the information processing device to generate, as abnormal notification information to be transmitted to a terminal of a user, feedback information including an action guideline relating to improvement of a health state of mind and body, the feedback information being acquired from the generative AI model as a response to the prompt sentence, and to transmit the abnormal notification information to the terminal; and
cause the terminal to display the abnormal notification information on a display device so that the user can confirm the abnormal mental and physical state and the action guideline.

### (Supplementary 2)

The system according to supplementary 1,
wherein the processor is configured to
cause the information processing device to extract, from the preprocessed digital data, feature quantities including at least statistical quantities and time-variation quantities for each predetermined time window, to input the feature quantities to the machine learning model to calculate the mental and physical state indices, and to include the feature quantities and the mental and physical state indices in the prompt sentence to be provided to the generative AI model.

### (Supplementary 3)

The system according to supplementary 1,
wherein the processor is configured to
cause the information processing device to acquire, from the storage device, past biological information histories and past mental and physical state index histories for each user, to derive baseline states and trend information based on the histories, and to add the baseline states and the trend information to the prompt sentence so that the generative AI model generates user-specific customized feedback information.

### Application Example 2

### (Supplementary 1)

A system comprising a processor,
wherein the processor is configured to
acquire biological information from a detection device and convert the biological information into digital data,
transmit the digital data to an information processing apparatus via a communication network,
store the digital data in a storage device of the information processing apparatus and analyze the digital data by a statistical processing method to generate a biological state indicator and an abnormality determination result,
input audio information, image information, and character information into an emotion recognition processing apparatus of the information processing apparatus and identify an emotional state by using an emotion recognition model,
integrate the biological state indicator and the emotional state in the information processing apparatus to calculate a risk level of a physical and mental state and determine necessity of issuing a warning based on the risk level,
generate, in the information processing apparatus, a prompt sentence including a state description based on the biological state indicator, the emotional state, and the necessity of issuing the warning, and input the prompt sentence into a generative artificial intelligence model to instruct the generative artificial intelligence model to generate feedback information and notification text,
transmit, from the information processing apparatus, the feedback information obtained from the generative artificial intelligence model to a user terminal and cause the user terminal to display the feedback information,
transmit, from the information processing apparatus, third-party notification text obtained from the generative artificial intelligence model to a predetermined external contact, and
obtain, in the information processing apparatus, evaluation information from a user regarding the feedback information and update at least one of the prompt sentence and output conditions of the generative artificial intelligence model based on the evaluation information.

### (Supplementary 2)

The system according to supplementary 1,
wherein the processor is configured to cause the generative artificial intelligence model to analyze the prompt sentence by a natural language processing technique and generate the feedback information including an action guideline for improving a physical and mental health state, based on the risk level of the physical and mental state and history information of the user.

### (Supplementary 3)

The system according to supplementary 1,
wherein the processor is configured to adjust, based on the biological information, the emotional state, and the evaluation information from the user, at least one of constituent elements and weighting of the prompt sentence so that content of the feedback information generated by the generative artificial intelligence model is adaptively optimized for each user.

What is claimed is:

1. A system comprising:
circuitry configured to
receive, via a communication interface coupled to a packet-switched network, sensor data acquired by a detection apparatus and converted into digital data,
store the digital data as structured records in a storage device and retrieve, from the storage device, historical records associated with an identifier of a source of the sensor data,
execute statistical analysis on the digital data and the historical records, the statistical analysis comprising calculation of statistical values, time-series analysis, and anomaly detection, to generate evaluation data,
construct a prompt sentence as structured input that includes the evaluation data, attribute information associated with the identifier, and trend indicators derived from the historical records,
transmit the prompt sentence to a generative neural network model and receive, from the generative neural network model, feedback information generated in response to the prompt sentence,
transmit output data derived from the feedback information to a terminal device via the communication interface, and
store, in the storage device, a correspondence record that associates the feedback information with the prompt sentence and the evaluation data.

2. The system according to claim 1, wherein the circuitry is further configured to calculate, as the statistical values, at least an arithmetic mean, a variance, and a standard deviation for each data type in the historical records over a predetermined time window.

3. The system according to claim 2, wherein the time-series analysis comprises computing a moving average over a sliding window, a difference series between consecutive time intervals, and a regression slope indicating a directional change of a measured quantity over time.

4. The system according to claim 3, wherein the anomaly detection comprises computing, for a latest data point relative to a distribution of the historical records, a normalized deviation value, and comparing the normalized deviation value with a threshold stored as a configuration parameter in the storage device.

5. The system according to claim 4, wherein the evaluation data comprises, for each data type, a numerical summary, a categorical label selected from a set including within-expected-range, slightly-abnormal, and significantly-abnormal, and a trend classification selected from a set including increasing, decreasing, and stable.

6. The system according to claim 5, wherein the circuitry is further configured to generate, for at least one data type classified as significantly-abnormal, a notification message comprising the categorical label and the normalized deviation value, and transmit the notification message to an external contact address stored in association with the identifier.

7. The system according to claim 1, wherein the circuitry is further configured to construct the prompt sentence by inserting the evaluation data, the attribute information, and the trend indicators into a predetermined natural-language template according to a string-composition operation, and wherein the prompt sentence further includes an instruction specifying a response style and a maximum output length for the generative neural network model.

8. The system according to claim 7, wherein the circuitry is further configured to dynamically include or omit segments of the prompt sentence based on a severity derived from the anomaly detection, such that the prompt sentence varies in content according to which data types exhibit anomalous values.

9. The system according to claim 8, wherein the generative neural network model comprises a transformer architecture having a plurality of self-attention layers and feed-forward sublayers, and the circuitry is further configured to supply the prompt sentence as a tokenized input sequence and receive, from the generative neural network model, a sequence of output tokens decoded into the feedback information.

10. The system according to claim 9, wherein the circuitry is further configured to perform post-processing on the feedback information, the post-processing comprising at least one of truncating the feedback information to a maximum length, removing disallowed phrases according to a pattern-matching filter, and enforcing inclusion of structural elements in the feedback information.

11. The system according to claim 1, wherein, before executing the statistical analysis, the circuitry is further configured to preprocess the digital data by complementing missing values in the structured records using interpolation and removing outlier values that deviate from a physiologically plausible range.

12. The system according to claim 11, wherein the circuitry is further configured to partition the preprocessed digital data into predetermined time windows, and for each time window, extract feature quantities comprising statistical quantities including mean, median, standard deviation, minimum, and maximum, and time-variation quantities including first-order differences and slopes computed by regression.

13. The system according to claim 12, wherein the circuitry is further configured to input the feature quantities into a trained machine learning model to calculate state indices, normalize the feature quantities using scaling parameters derived during training, and compare the state indices with preset threshold values to determine presence or absence of an abnormal state based on whether an index exceeding a threshold continues for a predetermined number of consecutive time windows.

14. The system according to claim 13, wherein the circuitry is further configured to derive, from past evaluation data and past state index records stored in the storage device, baseline values representing typical conditions for the identifier, and trend information indicating directional changes over a recent period, and add the baseline values and the trend information to the prompt sentence.

15. The system according to claim 1, wherein the circuitry is further configured to receive, from the terminal device, evaluation information expressing a rating of the feedback information, store the evaluation information in the storage device in association with the prompt sentence, and update at least one of constituent elements of the prompt sentence and output conditions of the generative neural network model based on the evaluation information.

16. The system according to claim 15, wherein the circuitry is further configured to apply an emotion recognition model to at least one of audio data, image data, and text data received from the terminal device to identify an emotional state, integrate the evaluation data and the emotional state to compute a risk level, and determine necessity of issuing a notification based on the risk level.

17. The system according to claim 16, wherein the sensor data comprises biological information including at least heart rate, blood pressure, sleep duration, and physical activity amount acquired from a wearable sensor device, and the state indices comprise a stress index and a sleep index representing respective aspects of a mental and physical condition of a user.

18. A system comprising:
circuitry configured to
receive, via a communication interface coupled to a packet-switched network, sensor data acquired by a detection apparatus and converted into digital data representing time-stamped measurements,
store the digital data as structured records in a storage device, each record comprising an identifier field, a measurement type field, a value field, a unit field, and a timestamp field,
retrieve, from the storage device, historical records associated with the identifier over a configurable time window,
preprocess the digital data and the historical records by complementing missing values and removing outlier values,
extract feature quantities for each predetermined sub-window, the feature quantities comprising statistical quantities and time-variation quantities,
input the feature quantities into a trained machine learning model to calculate state indices and compare the state indices with threshold values to detect an abnormal state that persists for a predetermined duration,
construct a prompt sentence that embeds the feature quantities, the state indices, attribute information, baseline values, and trend indicators into a natural-language template,
transmit the prompt sentence to a generative neural network model comprising a transformer architecture and receive feedback information, and
transmit output data derived from the feedback information to a terminal device and store, in the storage device, a correspondence record associating the feedback information with the prompt sentence and the state indices.

19. The system according to claim 18, wherein the circuitry is further configured to apply a multimodal emotion recognition model to at least one of audio data, image data, and text data to identify an emotional state, integrate the state indices and the emotional state using a fusion function to compute a risk level, generate a notification message for an external contact when the risk level exceeds a threshold, and receive evaluation information from a user to update at least one of the prompt sentence and generation parameters of the generative neural network model.

20. A method comprising:
receiving, by circuitry via a communication interface coupled to a packet-switched network, sensor data acquired by a detection apparatus and converted into digital data;
storing the digital data as structured records in a storage device and retrieving, from the storage device, historical records associated with an identifier of a source of the sensor data;
executing, by the circuitry, statistical analysis on the digital data and the historical records, the statistical analysis comprising calculation of statistical values, time-series analysis, and anomaly detection, to generate evaluation data;
constructing a prompt sentence as structured input that includes the evaluation data, attribute information associated with the identifier, and trend indicators derived from the historical records;
transmitting the prompt sentence to a generative neural network model and receiving, from the generative neural network model, feedback information generated in response to the prompt sentence;
transmitting output data derived from the feedback information to a terminal device via the communication interface; and
storing, in the storage device, a correspondence record that associates the feedback information with the prompt sentence and the evaluation data.
